(19) United States
(12) Patent Application Publication
(10) Pub. No.: US 2025/0375454 A1
(43) Pub. Date: Dec. 11, 2025

(54) METHODS FOR TREATING HEPATOCELLULAR CARCINOMA

(71) Applicant: THE HONG KONG POLYTECHNIC UNIVERSITY

(72) Inventors: Kin Wah LEE, Hong Kong (CN); Shakeel Ahmad KHAN, Hong Kong (CN); Huihai YANG, Hong Kong (CN)

(21) Appl. No.: 19/221,630

(22) Filed: May 29, 2025

Related U.S. Application Data
(60) Provisional application No. 63/657,193, filed on Jun. 7, 2024.

(51) Int. Cl. A61K 31/536; A61P 35/00

(52) U.S. Cl. CPC A61K 31/536; A61P 35/00

(57) ABSTRACT

A method of treating hepatocellular carcinoma in a subject in need thereof, the method including: administering a therapeutically effective amount of a benzoxazinone analog to the subject, wherein the benzoxazinone analogs can exhibit multi-targeted anticancer effects modulating molecular targets, such as c-Myc, EGFR, ERBB2, Bax, and Bcl-2, involved in hepatocellular carcinoma proliferation and survival.

### CROSS-REFERENCE TO RELATED APPLICATIONS

This application claims the benefit of priority of U.S. Provisional Application No. 63/657,193, filed on Jun. 7, 2024, the contents of which being hereby incorporated by reference in their entirety for all purposes.

### TECHNICAL FIELD

The present disclosure generally relates to the field of anticancer agents. More particularly, the present disclosure provides a method of treating hepatocellular carcinoma using benzoxazinone analogs.

### BACKGROUND

Hepatocellular carcinoma (HCC) is the most prevalent primary liver cancer and among the leading causes of cancer-related mortality worldwide. This malignancy is often associated with underlying chronic liver diseases, including hepatitis B and C infections, alcoholic liver disease, and nonalcoholic steatohepatitis. Despite advances in early detection and surgical interventions, the prognosis for HCC remains poor owing to its aggressive nature and high recurrence rates. Traditional chemotherapeutic approaches have limited efficacy, thus propelling the search for more effective treatment modalities. Molecular targeted therapy has revolutionized the therapeutic landscape for HCC by focusing on specific molecular targets that drive tumor growth and progression. These therapies aim to inhibit key signaling pathways implicated in HCC pathogenesis, such as the VEGF, PDGFR, and RAF/MEK/ERK pathways. Agents such as sorafenib and lenvatinib have demonstrated clinical benefits by improving overall survival and delaying disease progression in patients with advanced HCC. However, the survival benefit of sorafenib is modest due to the development of resistance and surprisingly, only 20% of patients tolerate sorafenib, resulting in moderate-to-severe adverse effects, necessitating the exploration of novel therapeutic strategies. Furthermore, the heterogeneity of HCC often renders single-target therapies insufficient, as they fail to address the multifaceted nature of disease mechanisms.

To overcome these limitations, there is increasing interest in polypharmacology, which aims to concurrently modulate multiple molecular targets. Polypharmacological agents can bind to and functionally influence several proteins, providing a holistic approach to disease management. This strategy can be achieved through either combination therapy or the development of single compounds capable of multiple target interactions. Polypharmacology offers several advantages over traditional combination therapies, including superior pharmacokinetic and safety profiles, a lower likelihood of acquired resistance, and streamlined treatment regimens that enhance patient compliance. The application of polypharmacology to molecular-targeted therapies is particularly promising. For instance, polypharmacological compounds have shown efficacy in treating KRAS mutant non-small cell lung cancers, which have proven refractory to conventional single-target agents. Despite these advancements, a significant challenge in polypharmacology remains the design of compounds that effectively inhibit multiple proteins with high potency. Traditionally, the discovery of such agents has been serendipitous, often requiring substantial time and resources to identify suitable hit scaffolds. However, recent progress in systems biology, system pharmacology, bioinformatics, machine learning, and computational modeling is beginning to address these challenges. These technologies facilitate the systematic prediction of compound-target interactions, and the identification of existing drugs with polypharmacological dual targeting potential.

Among the various chemical classes explored for their therapeutic potential, benzoxazinones have attracted attention due to their broad-spectrum biological activities, including anticancer, α-chymotrypsin antagonist, complement protein one receptor blocker, anti-cathepsin G, an inhibitor of human leukocyte elastase, anti-human coronavirus, antibacterial, antifungal, antiphlogistic. Drugs CX-614, Efavirenz, and Cetilistat contain benzoxazinone functionality in their molecular structures and have been developed for treating Parkinson's and Alzheimer's disease, AIDS, and obesity, respectively. Therefore, we hypothesized that incorporating benzoxazinones into the therapeutic portfolio for HCC, particularly within the framework of molecular-targeted therapy and polypharmacology, holds promise for developing comprehensive and effective treatment strategies.

### SUMMARY

Benzoxazinone derivatives were systematically evaluated to identify promising multi-target therapeutic candidates through an integrative polypharmacological approach. Specifically, it was determined whether these derivatives could effectively modulate multiple oncogenic targets pivotal to HCC pathogenesis using advanced computational techniques, including network pharmacology and molecular docking. Additionally, the most potent benzoxazinone derivatives were identified with favorable pharmacokinetic and toxicity profiles via ADMET screening. Furthermore, the therapeutic efficacy and safety of the benzoxazinone derivatives were evaluated in comparison to existing treatments through mechanistic studies, including in vitro cytotoxicity assays, western blot analysis, and in vivo evaluations using PLC/PRF/5 tumor-bearing and HCC patient-derived tumor xenograft (PDTX) mouse models. To accomplish these objectives, a focused library of benzoxazinone derivatives was computationally designed and screened, then experimentally validated their polypharmacological activities, and systematically identified the lead compound (ZAK-I-57) as a viable candidate for advanced therapeutic development.

Provided herein is a method of treating hepatocellular carcinoma in a subject in need thereof, the method comprising: administering a therapeutically effective amount of a compound to the subject, wherein the compound has Formula 1:

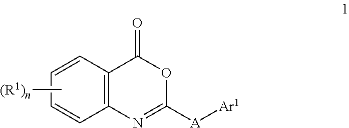

or a pharmaceutically acceptable salt thereof, wherein:
m is a whole number selected from 0-4;
A is —CH₂CH₂, —CH═CH—, —C≡C—, or absent;
Ar¹ is aryl or heteroaryl;
R¹ for each instance is independently alkyl, haloalkyl, perhaloalkyl, alkenyl, alkynyl, cycloalkyl, heterocycloalkyl, aryl, aralkyl, heteroaryl, nitrile, nitro, azido, —OR, —SR, —NR₂, —(C═O)R, —(C═O)OR, —(C═O)NR₂, —(C═NR)NR₂, —(S═O)R, —S(O)₂R, —S(O)₂OR, —S(O)₂NR₂, —(P═O)(OR)₂, or —(CR²₂)ₚY, wherein p for each occurrence is a whole number selected from 1-10; R² for each occurrence is independently selected from the group consisting of hydrogen, alkyl, alkenyl, alkynyl, cycloalkyl, heterocycloalkyl, aryl, and heteroaryl; and Y for each occurrence is selected from the group consisting of OR, SR, NR₂, —(C═O)R, —(C═O)OR, —O(C═O)R, —O(C═O)OR, —(C═O)NR₂, —(NR)(C═O)R, —(NR)(C═O)OR, —O(C═O)NR₂, —O(C═NR)NR₂, —(NR)(C═O)NR₂, —(C═NR)NR₂, —(NR)(C═NR)NR₂, —(S═O)R, —S(O)₂R, —S(O)₂OR, —S(O)₂NR₂, —OS(O)₂R, —(NR)S(O)₂R, —OS(O)₂OR, —OS(O)₂NR₂, —(NR)S(O)₂NR₂, —(NR)S(O)₂OR, and —(P═O)(OR)₂; and
R for each instance is independently selected from the group consisting of hydrogen, alkyl, haloalkyl, perhaloalkyl, alkenyl, alkynyl, cycloalkyl, heterocycloalkyl, aryl, heteroaryl, and aralkyl; or two instances of R together with the atom they are covalently bonded form a 3-6 membered cycloalkyl or heterocyloalkyl.

In certain embodiments, A is —CH═CH— or absent.

In certain embodiments, Ar¹ is a moiety selected from the group consisting of:

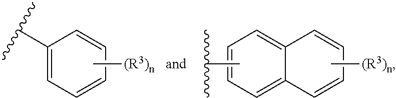

wherein n is a whole number selected from 0-4;
R³ for each instance is independently alkyl, haloalkyl, perhaloalkyl, alkenyl, alkynyl, cycloalkyl, heterocycloalkyl, aryl, aralkyl, heteroaryl, nitrile, nitro, azido, —OR, —SR, —NR₂, —(C═O)R, —(C═O)OR, —(C═O)NR₂, —(C═NR)NR₂, —(S═O)R, —S(O)₂R, —S(O)₂OR, —S(O)₂NR₂, —(P═O)(OR)₂, or —(CR²₂)ₚY, wherein p for each occurrence is a whole number selected from 1-10; R² for each occurrence is independently selected from the group consisting of hydrogen, alkyl, alkenyl, alkynyl, cycloalkyl, heterocycloalkyl, aryl, and heteroaryl; and Y for each occurrence is selected from the group consisting of OR, SR, NR₂, —(C═O)R, —(C═O)OR, —O(C═O)R, —O(C═O)OR, —(C═O)NR₂, —(NR)(C═O)R, —(NR)(C═O)OR, —O(C═O)NR₂, —O(C═NR)NR₂, —(NR)(C═O)NR₂, —(C═NR)NR₂, —(NR)(C═NR)NR₂, —(S═O)R, —S(O)₂R, —S(O)₂OR, —S(O)₂NR₂, —OS(O)₂R, —(NR)S(O)₂R, —OS(O)₂OR, —OS(O)₂NR₂, —(NR)S(O)₂NR₂, —(NR)S(O)₂OR, and —(P═O)(OR)₂.

In certain embodiments, n is 0 or 1 and R³ for each instance is independently alkyl, haloalkyl, perhaloalkyl, cycloalkyl, heterocycloalkyl, aryl, aralkyl, heteroaryl, nitrile, nitro, —OR, —SR, —NR₂, —(C═O)NR₂, —(NR)(C═O)R, or —(NR)(C═O)OR.

In certain embodiments, n is 0 or 1 and R³ for each instance is independently alkyl, halide, or —OR.

In certain embodiments, Ar¹ is selected from the group consisting of 4-bromophenyl, 4-hydroxy-phenyl, 2-methyl-phenyl, 3-chloro-phenyl, 4-fluoro-phenyl, 3,4-dimethoxy-phenyl, or 2-naphthyl.

In certain embodiments, R¹ for each instance is independently alkyl, haloalkyl, perhaloalkyl, cycloalkyl, heterocycloalkyl, aryl, aralkyl, heteroaryl, nitrile, nitro, —OR, —SR, —NR₂, —(C═O)NR₂, —(NR)(C═O)R, or —(NR)(C═O)OR.

In certain embodiments, at least one R¹ is nitro.

In certain embodiments, the compound has Formula 2:

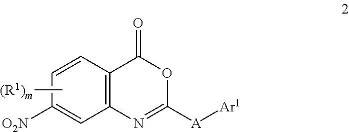

or a pharmaceutically acceptable salt thereof, wherein
m is a whole number selected from 0-2;
A is —CH═CH— or absent;
Ar¹ is a moiety selected from the group consisting of:

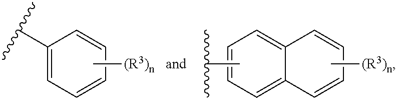

wherein n is a whole number selected from 0-2;
R¹ for each instance is independently alkyl, haloalkyl, perhaloalkyl, cycloalkyl, heterocycloalkyl, aryl, aralkyl, heteroaryl, nitrile, nitro, —OR, —SR, —NR₂, —(C═O)NR₂, —(NR)(C═O)R, or —(NR)(C═O)OR;
R³ for each instance is independently alkyl, haloalkyl, perhaloalkyl, cycloalkyl, heterocycloalkyl, aryl, aralkyl, heteroaryl, nitrile, nitro, —OR, —SR, —NR₂, —(C═O)NR₂, —(NR)(C═O)R, or —(NR)(C═O)OR; and
R for each instance is independently selected from the group consisting of hydrogen, alkyl, haloalkyl, perhaloalkyl, alkenyl, alkynyl, cycloalkyl, heterocycloalkyl, aryl, heteroaryl, and aralkyl; or two instances of R together with the atom they are covalently bonded form a 3-6 membered cycloalkyl or heterocyloalkyl.

In certain embodiments, at least one R³ is selected from the group consisting of halide, —OR, and alkyl.

In certain embodiments, Ar¹ is selected from the group consisting of 4-bromophenyl, 4-hydroxy-phenyl, 2-methyl-phenyl, 3-chloro-phenyl, 4-fluoro-phenyl, 3,4-dimethoxy-phenyl, and 2-naphthyl.

In certain embodiments, m is 0 and at least one R³ is selected from the group consisting of halide, —OR, and alkyl.

In certain embodiments, the compound is selected from the group consisting of:

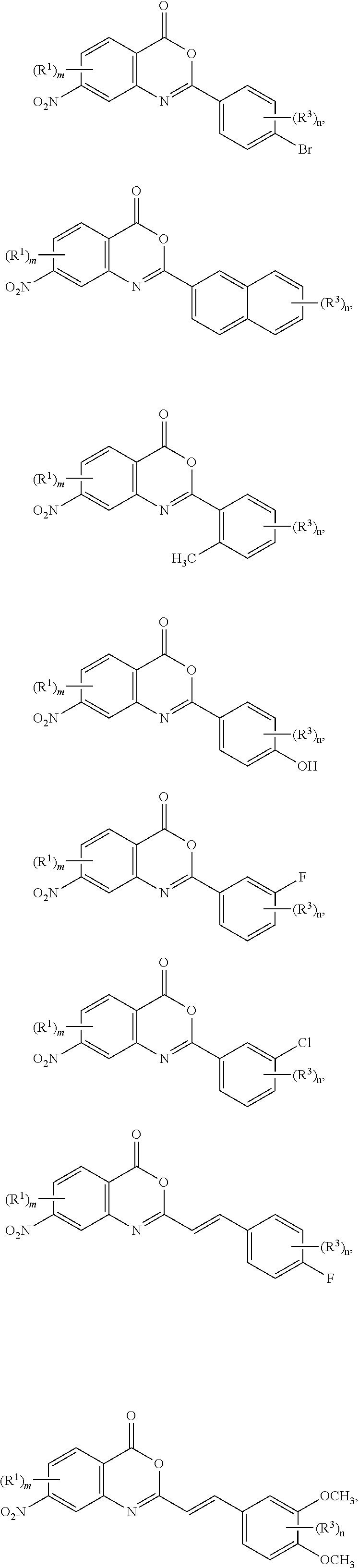

and
pharmaceutically acceptable salts thereof, wherein
m is a 0 or 1;
n is 0 or 1;
R¹ is alkyl, haloalkyl, perhaloalkyl, cycloalkyl, heterocycloalkyl, aryl, aralkyl, heteroaryl, nitrile, nitro, —OR, —SR, —NR₂, —(C═O)NR₂, —(NR)(C═O)R, or —(NR)(C═O)OR; and
R³ is alkyl, haloalkyl, perhaloalkyl, cycloalkyl, heterocycloalkyl, aryl, aralkyl, heteroaryl, nitrile, nitro, —OR, —SR, —NR₂, —(C═O)NR₂, —(NR)(C═O)R, or —(NR)(C═O)OR; and
R for each instance is independently selected from the group consisting of hydrogen, alkyl, cycloalkyl, heterocycloalkyl, aryl, heteroaryl, and aralkyl; or two instances of R together with the atom they are covalently bonded form a 3-6 membered cycloalkyl or heterocyloalkyl.

In certain embodiments, the compound is selected from the group consisting of:

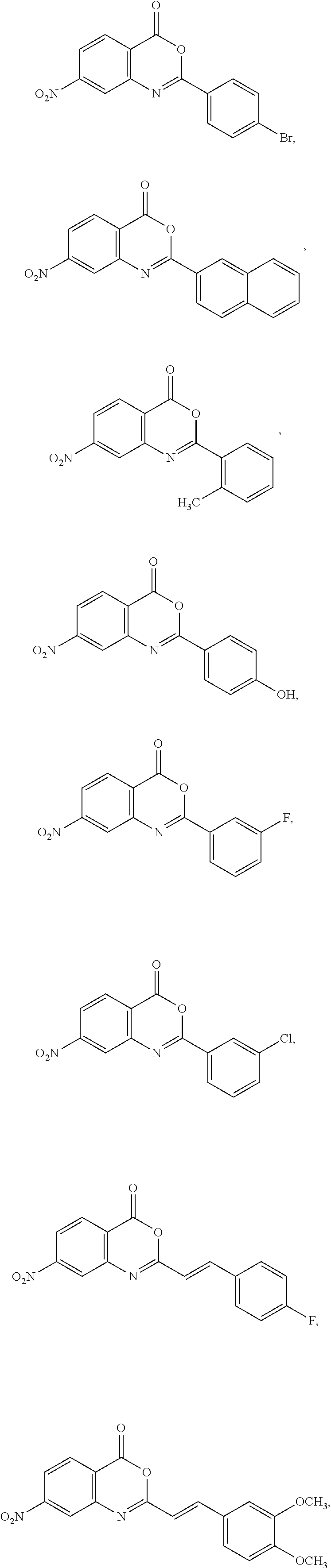

and
pharmaceutically acceptable salts thereof.

In certain embodiments, the compound is:

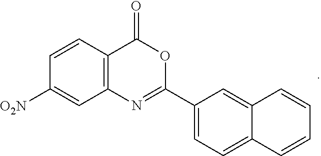 [SMILES: O=c1oc(-c2ccc3ccccc3c2)nc2cc([N+](=O)[O-])ccc12 ; "."]

In certain embodiments, administration of the compound reduces the expression of one or more oncogenic proteins in the hepatocellular carcinoma, wherein the one or more oncogenic proteins is selected from the group consisting of EGFR, c-Myc, and ERBB2.

In certain embodiments, administration of the compound results in at least one of an increase in expression of Bax and reduction in expression of Bcl-2 in the hepatocellular carcinoma.

In certain embodiments, the compound induces apoptosis in the hepatocellular carcinoma.

In certain embodiments, the compound exhibits selective cytotoxicity against the hepatocellular carcinoma.

In certain embodiments, the compound reduces tumor volume and weight in a PLC/PRF/5 xenograft model.

In certain embodiments, the compound reduces hepatocellular carcinoma progression in a patient-derived tumor xenograft (PDTX) model.

In certain embodiments, the compound demonstrates anti-hepatocellular carcinoma efficacy comparable to or greater than sorafenib.

In certain embodiments, the administration of the compound results in suppression of hepatocellular carcinoma proliferation as measured by Ki-67 expression.

In certain embodiments, the compound does not induce observable histopathological alterations in the liver, kidney, heart, lung, or spleen of the subject, as determined by hematoxylin and eosin (H&E) staining.

### BRIEF DESCRIPTION OF THE DRAWINGS

The foregoing aspects and many of the attendant advantages of this invention will become more readily appreciated and understood by reference to the following detailed description, when taken in conjunction with the accompanying drawings.

FIG. 1. DFT study. (A) Optimized geometry, (B) HOMO−1, (C) HOMO, (D) LUMO, and (E) LUMO+1 of ZAAK-I-57. HOMO−1 orbitals are located all over ZAK-I-57. The HOMO orbitals were located on the nitro group and core of ZAK-I-57. The LUMO and LUMO+1 orbitals were located all over ZAK-I-57. (F) MEP mapping. Green, orange, blue, red, and yellow on the MEP surfaces indicate the order of magnitude of the electrostatic potential throughout the structures. The colors were arranged in increasing order: red>orange>yellow>green>blue. (G) Mulliken charges of ZAK-I-57.

FIG. 2. Molecular docking interactions of ZAK-I-57 and ZAK-I-64 with key oncogenic proteins. ZAK-I-57 binds to (A) c-Myc (B) ESR1, (C) EGFR, (D) CCND1, and (E) ERBB2. ZAK-I-64 binds (F) HSP90AA1.

FIG. 3. (A-F) cell viability percentage of Huh7 and PLC/PRF/5 cell lines treated with ZAK-I-57 (24 h), ZAK-I-64 (48 h), and ZAK-I-68 (48 h). (G) Western blotting results. EGFR, c-Myc, and ERBB2 expression levels after treatment with ZAK-I-57 (48 h), ZAK-I-64 (72 h), and ZAK-I-68 (72 h).

FIG. 4. Antitumor efficacy of ZAK-I-57 in PLC/PRF/5 tumor-bearing mice compared with sorafenib and vehicle control. (A) Body weight change curves of the mice [Statistical analysis: p>0.05 (Control vs Sorafenib at 30 mg/kg; Control vs. ZAK-I-57 at 15 mg/kg; Control vs. ZAK-I-57 at 30 mg/kg)]. (B and C) Tumor volume change curves [Statistical analysis: ***p=0.0004 (Control vs Sorafenib at 30 mg/kg); ****p<0.0001 (Control vs. ZAK-I-57 at 15 mg/kg and 30 mg/kg)] and photographs of tumors, respectively. Scale bar=1 cm. (D) Tumor weight change of the mice. [Statistical analysis: ns=p=0.3935 (Control vs Sorafenib at 30 mg/kg); ns=p=0.0776 (Control vs. ZAK-I-57 at 15 mg/kg); *p=0.0320 (Control vs. ZAK-I-57 at 30 mg/kg)]. (E) Percentage of tumor growth inhibition (n=5 per group). [Statistical analysis: *p=0.0241 (Control vs Sorafenib at 30 mg/kg); *p=0.0308 (Control vs. ZAK-I-57 at 15 mg/kg); **p=0.0022 (Control vs. ZAK-I-57 at 30 mg/kg)]. (F) western blotting results for three representative PLC/PRF/5 tumor-bearing mice from each group. Relative quantitative expression levels of (G) EGFR (ns=p=0.4777; *p=0.0339), (H) c-Myc (ns=p=0.0609; *p=0.0195), (I) Bax (ns=p=0.4673; ****p<0.0001), and (J) Bcl-2 (ns=p=0.4757; *p=0.0180) in PLC/PRF/5 tumor-bearing mice treated with ZAK-I-57 (15 mg/kg and 30 mg/kg) compared to the control.

FIG. 5. Antitumor efficacy of ZAK-I-57 in PDTX mouse model compared to sorafenib and vehicle control. (A) Schematic representation of the PDTX mouse model (n=5 per group) (Created in BioRender.com). (B) The body weight change curve of the mice [Statistical analysis: p>0.05 (Control vs Sorafenib at 30 mg/kg; Control vs. ZAK-I-57 at 15 mg/kg; Control vs. ZAK-I-57 at 30 mg/kg)]. (C and D) Tumor volume change curves [Statistical analysis: ****p<0.0001 (Control vs Sorafenib at 30 mg/kg; Control vs. ZAK-I-57 at 15 mg/kg; Control vs. ZAK-I-57 at 30 mg/kg)] and photographs of tumors, respectively. Scale bar=1 cm. (E) Tumor weight change of the mice [Statistical analysis: ns=0.0856 (Control vs. Sorafenib at 30 mg/kg); *p=0.0204 (Control vs. ZAK-I-57 at 15 mg/kg); *p=0.0324 (Control vs. ZAK-I-57 at 30 mg/kg)]. (F) Percentage of tumor growth inhibition [Statistical analysis: ns=p=0.3062 (Control vs Sorafenib at 30 mg/kg); *p=0.0218 (Control vs. ZAK-I-57 at 15 mg/kg); *p=0.0273 (Control vs. ZAK-I-57 at 30 mg/kg)]. (G) western blotting results for four representative PDTX mouse models from each group. Relative quantitative expression levels of (H) EGFR (ns=p=0.1267; ****p<0.0001), (I) c-Myc (ns=p=0.6023; *p=0.0496), (J) Bax (ns=p=0.1040; *p=0.0103), and (K) Bcl-2 (ns=p=0.5331; *p=0.0491) in PDTX mouse models.

FIG. 6. Histopathological Assessment. H&E staining of (A) PLC/PRF/5 tumor-bearing mouse tissues and (B) PDTX mouse model tissues treated with sorafenib (30 mg/kg) and ZAK-I-57 at two doses (15 and 30 mg/kg) compared with vehicle control. Ki-67 staining of (C) PLC/PRF/5 tumor-bearing mouse tissues and (D) PDTX mouse model tissues treated with sorafenib (30 mg/kg) and ZAK-I-57 at two doses (15 and 30 mg/kg) compared with the vehicle control. Scale bar=20 μm. Arrows indicates Ki-67⁺ cells. Quantitative determination of the number of Ki67⁺ stained cells in (E) PLC/PRF/5 tumor-bearing mouse tissues [Statistical analysis: *p=0.0120 (Control vs. Sorafenib at 30 mg/kg); *p=0.0431 (Control vs. ZAK-I-57 at 15 mg/kg); ***p=0.0008 (Control vs. ZAK-I-57 at 30 mg/kg)] and (F) PDTX mouse model tissues (***p=0.0005; ****p<0.0001) treated with sorafenib (30 mg/kg) and ZAK-I-57 at two doses (15 and 30 mg/kg) compared with vehicle control, respectively.

FIG. 7. Biosafety profile assessment. (A) Gross anatomical assessment of vital organs (heart, liver, kidney, spleen, and lungs) from a PLC/PRF/5 tumor-bearing mouse model administered a control vehicle, sorafenib at a therapeutic dose of 30 mg/kg, and ZAK-I-57 at two doses (15 and 30 mg/kg). Scale bar=1 cm. (B) Histopathological compendium delineates H&E-stained organ sections from a PLC/PRF/5 tumor-bearing mouse model administered a control vehicle, sorafenib at a therapeutic dose of 30 mg/kg, and ZAK-I-57 at two doses (15 and 30 mg/kg). Scale bar=20 μm.

FIG. 8. (A) Structural representation of ZAK-I-57 and its core characteristics. The key pharmacological attributes of the compounds are highlighted in the upper right, showing drug-likeness, hit-likeness, high oral bioavailability, and other pharmacological properties. (B) Mechanistic pathway of ZAK-I-57: ZAK-I-57 inhibits EGFR, suppresses the MEK/MAPK signaling pathway, downregulates Bcl-2, and upregulates Bax in the mitochondria, thereby promoting apoptosis. Additionally, it inhibits the oncogenic transcription factor c-Myc, thereby enhancing its antiproliferative and pro-apoptotic effects.

FIG. 9. Scheme for the synthesis of benzoxazinone derivatives.

FIG. 10. Optimized geometry diagrams of ZAK-I-55, ZAK-I-64, ZAK-I-68, ZAK-I-87, ZAK-I-90, ZAK-I-93, and ZAK-I-97.

FIG. 11. HOMO−1, HOMO, LUMO, and LUMO+1 orbitals of ZAK-I-55, ZAK-I-64, ZAK-I-68, ZAK-I-87, ZAK-I-90, ZAK-I-93, and ZAK-I-97.

FIG. 12. MEP mapping of ZAK-I-55, ZAK-I-64, ZAK-I-68, ZAK-I-87, ZAK-I-90, ZAK-I-93, and ZAK-I-97.

FIG. 13. Mulliken charges spectra of (A-F) ZAK-I-55, ZAK-I-64, ZAK-I-68, ZAK-I-87, ZAK-I-90, ZAK-I-93, and ZAK-I-97.

FIG. 14. (A) Potential protein targets of benzoxazinone derivatives and HCC-related targets. (B) Intersecting targets between HCC-related targets and potential protein targets of benzoxazinone derivatives. (C) A PPI network of 50 intersecting targets was constructed using the STRING database. (D) PPI network of 50 intersecting targets and (E) 23 potential anti-HCC core targets were constructed using Cytoscape software. DC denotes degree of centrality.

FIG. 15. (A) The 23 anti-HCC core targets were ranked by a DC value greater than the average value (31.83). (B) Expression of anti-HCC core targets in the LHIC and normal samples.

FIG. 16. (A) Network of eight benzoxazinone derivatives with 50 anti-HCC targets. (B) Hub network between eight benzoxazinone derivatives and 23 anti-HCC core targets. (C) Eight benzoxazinone derivatives with respect to their degree values in hub network.

FIG. 17. GO enrichment analysis of 50 anti-HCC targets implicated in HCC treatment with benzoxazinone derivatives.

FIG. 18. Top 30 KEGG signaling pathways involved in the anti-HCC therapeutic actions of benzoxazinone derivatives.

FIG. 19. MIHA cell viability following (A) 24 h and (B) 48 h treatments with ZAK-I-57. Viability remained above 90% across all concentrations, yielding an IC₅₀>100 μM and indicating minimal cytotoxicity in normal hepatocytes.

FIG. 20. Biosafety profile assessment. (A) Gross anatomical assessment of vital organs (hearts, livers, kidneys, spleens, and lungs) from an HCC PDTX mouse model administered a control vehicle, sorafenib at a therapeutic dose of 30 mg/kg, and ZAK-I-57 at two doses (15 and 30 mg/kg). Scale bar=1 cm (B) Histopathological compendium delineates H&E-stained organ sections from an HCC PDTX mouse model administered a control vehicle, sorafenib at a therapeutic dose of 30 mg/kg, and ZAK-I-57 at two doses (15 and 30 mg/kg). Scale bar=20 μm.

FIG. 21. Reactivity indices for the benzoxazinone derivatives.

FIG. 22. Optimized geometrical parameters of ZAK-I-55.

FIG. 23. Optimized geometrical parameters of ZAK-1-57.

FIG. 24. Optimized geometrical parameters of ZAK-1-64.

FIG. 25. Optimized geometrical parameters of ZAK-1-68.

FIG. 26. Optimized geometrical parameters of ZAK-I-87.

FIG. 27. Optimized geometrical parameters of ZAK-I-90.

FIG. 28. Optimized geometrical parameters of ZAK-J-93,

FIG. 29. Optimized geometrical parameters of ZAK-I-97.

FIG. 30. Molecular orbital energies and other properties of the benzoxazinone derivatives.

FIG. 31. Second-order perturbance theory analysis of NBO charges of ZAK-I-55.

FIG. 32. Second-order perturbance theory analysis of NBO charges of ZAK-I-57.

FIG. 33. Second-order perturbance theory analysis of NBO charges of ZAK-I-64.

FIG. 34. Second-order perturbance theory analysis of NBO charges of ZAK-I-68.

FIG. 35. Second-order perturbance theory analysis of NBO charges of ZAK-I-87.

FIG. 36. Physicochemical properties of benzoxazinone derivatives.

FIG. 37. Drug-likeness and medicinal chemistry properties of benzoxazinone derivatives.

FIG. 38. Pharmacokinetic properties of the benzoxazinone derivatives.

FIG. 39. Intersecting targets between HCC-related and potential protein targets of benzoxazinone derivatives.

FIG. 40. The molecular docking results were expressed in the form of binding energies.

FIG. 41. Comparative analysis of different physiochemical properties between ZAK-I-57 and Sorafenib.

### DETAILED DESCRIPTION

### Definitions

The following terms shall be used to describe the present invention. In the absence of a specific definition set forth herein, the terms used to describe the present invention shall be given their common meaning as understood by those of ordinary skill in the art.

As used herein, the terms “treat”, “treating”, “treatment”, and the like refer to reducing or ameliorating a disorder/disease and/or symptoms associated therewith. It will be appreciated, although not precluded, treating a disorder or condition does not require that the disorder, condition, or symptoms associated therewith be completely eliminated. In certain embodiments, treatment includes prevention of a disorder or condition, and/or symptoms associated therewith. The term “prevention” or “prevent” as used herein refers to any action that inhibits or at least delays the development of a disorder, condition, or symptoms associated therewith. Prevention can include primary, secondary and tertiary prevention levels, wherein: a) primary prevention avoids the development of a disease; b) secondary prevention activities are aimed at early disease treatment, thereby increasing opportunities for interventions to prevent progression of the disease and emergence of symptoms; and c) tertiary prevention reduces the negative impact of an already established disease by restoring function and reducing disease-related complications.

The term “subject” as used herein, refers to an animal, typically a mammal or a human, that will be or has been the object of treatment, observation, and/or experiment. When the term is used in conjunction with administration of a compound described herein, then the subject has been the object of treatment, observation, and/or administration of the compound described herein.

The term “therapeutically effective amount” as used herein, means that amount of the compound or pharmaceutical agent that elicits a biological and/or medicinal response in a cell culture, tissue system, subject, animal, or human that is being sought by a researcher, veterinarian, clinician, or physician, which includes alleviation of the symptoms of the disease, condition, or disorder being treated.

The term “hepatocellular carcinoma” as used herein refers to cancer that arises from hepatocytes, the major cell type of the liver.

The terms “overexpressed”, “overexpression”, and “overexpressing” as used herein, may be taken to mean the expression (i.e., level/amount) of mRNA and/or protein found in a particular cell (i.e., a cancer cell) is elevated compared to the expression (i.e., level/amount) of mRNA and/or protein found in a normal, healthy cell (i.e., a cancer-free cell). Thus, for example, a “Bcl-2 overexpressing cancer” will be understood to be a cancer which expresses elevated RNA transcript and/or protein levels of Bcl-2 compared to the RNA transcript and/or protein levels of Bcl-2 found in normal, healthy cells. The Bcl-2 overexpressing cancer can be a cancer having levels of RNA transcript and/or protein of Bcl-2 at least 25% greater than the RNA transcript and/or protein levels of Bcl-2 in a normal, healthy cell. In certain embodiments, the Bcl-2 overexpressing cancer is a cancer having levels of RNA transcript and/or protein of MYC at least 50% greater, at least 100% greater, at least 200% greater, or at least 400% greater than the RNA transcript and/or protein levels of Bcl-2 in a normal, healthy cell. The level of expression can be determined by any suitable means known in the art. For example, the level of expression of Bcl-2 can be determined by measuring Bcl-2 protein levels. The Bcl-2 protein levels may be measured using any suitable technique known in the art, such as, SDS-PAGE followed by Western blot using suitable antibodies raised against the target protein. In addition, or alternatively, the level of expression of Bcl-2 may be determined by measuring the level of mRNA.

As used herein, unless otherwise indicated, the term “halo” or “halide” includes fluoro, chloro, bromo or iodo. Preferred halo groups are fluoro, chloro and bromo.

The term “alkyl” is art-recognized, and includes saturated aliphatic groups, including straight-chain alkyl groups, branched-chain alkyl groups, cycloalkyl (alicyclic) groups, alkyl substituted cycloalkyl groups, and cycloalkyl substituted alkyl groups. In certain embodiments, a straight chain or branched chain alkyl has about 30 or fewer carbon atoms in its backbone (e.g., C₁-C₃₀ for straight chain, C₃-C₃₀ for branched chain), and alternatively, about 20 or fewer.

As used herein, “cycloalkyl” by itself or as part of another substituent means, unless otherwise stated, a monocyclic hydrocarbon having between 3-12 carbon atoms in the ring system and includes hydrogen, straight chain, branched chain, and/or cyclic substituents. Exemplary cycloalkyls include cyclopropyl, cyclobutyl, cyclopentyl, cyclohexyl, cycloheptyl, and the like.

As used herein, unless otherwise indicated, the term “alkenyl”, as used herein, unless otherwise indicated, includes alkyl groups as defined above having at least one carbon-carbon double bond at some point in the alkyl chain.

As used herein, unless otherwise indicated, the term “alkynyl”, as used herein, unless otherwise indicated, includes alkyl groups as defined above having at least one carbon-carbon triple bond at some point in the alkyl chain.

The term “aralkyl” is art-recognized and refers to an alkyl group substituted with an aryl group (e.g., an aromatic or heteroaromatic group).

The term “aryl” as used herein include substituted or unsubstituted single-ring aromatic groups in which each atom of the ring is carbon. Preferably the ring is a 5- to 10-membered ring, more preferably a 6- to 10-membered ring or a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like. The aryl group can be optionally substituted. Exemplary substitution on an aryl group can include, for example, a halogen, a haloalkyl such as trifluoromethyl, a hydroxyl, a carbonyl (such as a carboxyl, an alkoxycarbonyl, a formyl, or an acyl such as an alkylC(O)), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a silyl ether, a sulfhydryl, an alkylthio, a sulfate, a sulfonate, a sulfamoyl, a sulfonamido, a sulfonyl, a heterocyclyl, an aralkyl, or an aromatic or heteroaromatic moiety. The aromatic ring may be substituted at one or more ring positions with such substituents as described above, for example, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings may be cycloalkyls, cycloalkenyls, cycloalkynyls, aryls and/or heterocyclyls.

The term “heterocycloalkyl” or “heterocyclyl” as used herein includes reference to a saturated heterocyclic moiety having 3, 4, 5, 6 or 7 ring carbon atoms and 1, 2, 3, 4 or 5 ring heteroatoms selected from nitrogen, oxygen, phosphorus and sulfur. The group may be a polycyclic ring system but more often is monocyclic. This term includes reference to groups such as azetidinyl, pyrrolidinyl, tetrahydrofuranyl, piperidinyl, oxiranyl, pyrazolidinyl, imidazolyl, indolizidinyl, piperazinyl, thiazolidinyl, morpholinyl, thiomorpholinyl, quinolizidinyl and the like.

As used herein, “heteroaryl” refers to an aromatic monocyclic ring system containing at least one ring heteroatom selected from oxygen (O), nitrogen (N), sulfur (S), silicon (Si), and selenium (Se) or a polycyclic ring system where at least one of the rings present in the ring system is aromatic and contains at least one ring heteroatom. Polycyclic heteroaryl groups include those having two or more heteroaryl rings fused together, as well as those having at least one monocyclic heteroaryl ring fused to one or more aromatic carbocyclic rings, non-aromatic carbocyclic rings, and/or non-aromatic cycloheteroalkyl rings. A heteroaryl group, as a whole, can have, for example, 5 to 24 ring atoms and contain 1-5 ring heteroatoms (i.e., 5-20 membered heteroaryl group). The heteroaryl group can be attached to the defined chemical structure at any heteroatom or carbon atom that results in a stable structure. Generally, heteroaryl rings do not contain O—O, S—S, or S—O bonds. However, one or more N or S atoms in a heteroaryl group can be oxidized (e.g., pyridine N-oxide thiophene S-oxide, thiophene S,S-dioxide). Examples of heteroaryl groups include, for example, the 5- or 6-membered monocyclic and 5-6 bicyclic ring systems shown below:

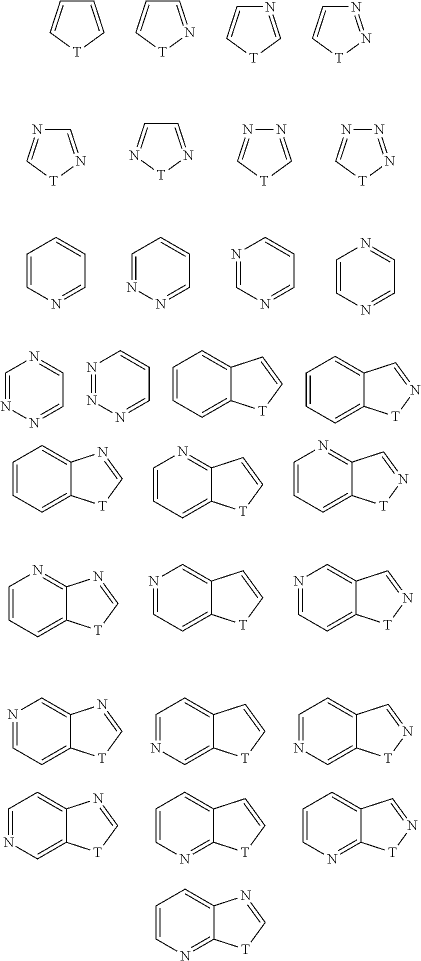

where T is O, S, NH, N-alkyl, N-aryl, N-(arylalkyl) (e.g., N-benzyl), SiH₂, SiH(alkyl), Si(alkyl)₂, SiH(arylalkyl), Si(arylalkyl)₂, or Si(alkyl)(arylalkyl). Examples of such heteroaryl rings include pyrrolyl, furyl, thienyl, pyridyl, pyrimidyl, pyridazinyl, pyrazinyl, triazolyl, tetrazolyl, pyrazolyl, imidazolyl, isothiazolyl, thiazolyl, thiadiazolyl, isoxazolyl, oxazolyl, oxadiazolyl, indolyl, isoindolyl, benzofuryl, benzothienyl, quinolyl, 2-methylquinolyl, isoquinolyl, quinoxalyl, quinazolyl, benzotriazolyl, benzimidazolyl, benzothiazolyl, benzisothiazolyl, benzisoxazolyl, benzoxadiazolyl, benzoxazolyl, cinnolinyl, 1H-indazolyl, 2H-indazolyl, indolizinyl, isobenzofuyl, naphthyridinyl, phthalazinyl, pteridinyl, purinyl, oxazolopyridinyl, thiazolopyridinyl, imidazopyridinyl, furopyridinyl, thienopyridinyl, pyridopyrimidinyl, pyridopyrazinyl, pyridopyridazinyl, thienothiazolyl, thienoxazolyl, thienoimidazolyl groups, and the like. Further examples of heteroaryl groups include 4,5,6,7-tetrahydroindolyl, tetrahydroquinolinyl, benzothienopyridinyl, benzofuropyridinyl groups, and the like. In certain embodiments, heteroaryl groups can be substituted as described herein. In certain embodiments, heteroaryl groups can be optionally substituted.

The term “optionally substituted” refers to a chemical group, such as alkyl, cycloalkyl aryl, and the like, wherein one or more hydrogen may be replaced with a substituent as described herein, for example, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like

As used herein, “heteroatom” refers to an atom of any element other than carbon or hydrogen and includes, for example, nitrogen, oxygen, silicon, sulfur, phosphorus, and selenium.

The term “nitro” is art-recognized and refers to —NO₂; the term “halogen” is art-recognized and refers to —F, —Cl, —Br or —I; the term “sulfhydryl” is art-recognized and refers to —SH; the term “hydroxyl” means —OH; and the term “sulfonyl” and “sulfone” is art-recognized and refers to —SO₂—. “Halide” designates the corresponding anion of the halogens.

The symbol “ [symbol] ” or “ [symbol] ” or “ [symbol] ” or “ [symbol] ” in a chemical structure represents a position from where the specified chemical structure is bonded to another chemical structure.

As used herein, the term “pharmaceutically acceptable salt” refers to those salts which are, within the scope of sound medical judgment, suitable for use in contact with the tissues of subjects without undue toxicity, irritation, allergic response and the like, and are commensurate with a reasonable benefit/risk ratio. Pharmaceutically acceptable salts are well known in the art. For example, Berge et al. describes pharmaceutically acceptable salts in detail in J. Pharmaceutical Sciences (1977) 66:1-19. Pharmaceutically acceptable salts of the compounds provided herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, besylate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. In certain embodiments, organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like.

Pharmaceutically acceptable salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, iron, zinc, copper, manganese, aluminum, and the like. Further pharmaceutically acceptable salts include, when appropriate, non-toxic ammonium, quaternary ammonium, and amine cations formed using counterions, such as halide, hydroxide, carboxylate, sulfate, phosphate, nitrate, lower alkyl sulfonate, and aryl sulfonate. Organic bases from which salts can be derived include, for example, primary, secondary, and tertiary amines, substituted amines including naturally occurring substituted amines, cyclic amines, basic ion exchange resins, and the like, such as isopropylamine, trimethylamine, diethylamine, triethylamine, tripropylamine, and ethanolamine. In certain embodiments, the pharmaceutically acceptable base addition salt is chosen from ammonium, potassium, sodium, calcium, and magnesium salts.

Provided herein is a method of treating hepatocellular carcinoma in a subject in need thereof, the method comprising: administering a therapeutically effective amount of a compound to the subject, wherein the compound has Formula 1:

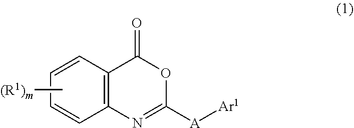

or a pharmaceutically acceptable salt thereof, wherein:
m is a whole number selected from 0-4;
A is —CH₂CH₂, —CH═CH—, —C≡C—, or absent, wherein —CH═CH— is an (E)-alkene or a (Z)-alkene;
Ar¹ is aryl or heteroaryl;
R¹ for each instance is independently alkyl, haloalkyl, perhaloalkyl, alkenyl, alkynyl, cycloalkyl, heterocycloalkyl, aryl, aralkyl, heteroaryl, nitrile, nitro, azido, —OR, —SR, —NR₂, —(C═O)R, —(C═O)OR, —(C═O)NR₂, —(C═NR)NR₂, —(S═O)R, —S(O)₂R, —S(O)₂OR, —S(O)₂NR₂, —(P═O)(OR)₂, or —(CR²₂)ₚY, wherein p for each occurrence is a whole number selected from 1-10; R² for each occurrence is independently selected from the group consisting of hydrogen, alkyl, alkenyl, alkynyl, cycloalkyl, heterocycloalkyl, aryl, and heteroaryl; and Y for each occurrence is selected from the group consisting of OR, SR, NR₂, —(C═O)R, —(C═O)OR, —O(C═O)R, —O(C═O)OR, —(C═O)NR₂, —(NR)(C═O)R, —(NR)(C═O)OR, —O(C═O)NR₂, —O(C═NR)NR₂, —(NR)(C═O)NR₂, —(C═NR)NR₂, —(NR)(C═NR)NR₂, —(S═O)R, —S(O)₂R, —S(O)₂OR, —S(O)₂NR₂, —OS(O)₂R, —(NR)S(O)₂R, —OS(O)₂OR, —OS(O)₂NR₂, —(NR)S(O)₂NR₂, —(NR)S(O)₂OR, and —(P═O)(OR)₂; and
R for each instance is independently selected from the group consisting of hydrogen, alkyl, haloalkyl, perhaloalkyl, alkenyl, alkynyl, cycloalkyl, heterocycloalkyl, aryl, heteroaryl, and aralkyl; or two instances of R together with the atom they are covalently bonded form a 3-6 membered cycloalkyl or heterocyloalkyl.

In certain embodiments, m is a whole number selected from 0-3, 0-2, or 0-1, In certain embodiments, m is a whole number selected from 0, 1, 2, 3, or 4.

In certain embodiments, n is a whole number selected from 0-3, 0-2, or 0-1, In certain embodiments, n is a whole number selected from 0, 1, 2, 3, or 4.

In certain embodiments, p is a whole number selected from 1-8, 1-6, 1-4, or 1-2.

A can be —CH═CH— or absent. In instances in which A is absent, Ar1 is covalently bonded directly to the oxazinone ring system as shown below:

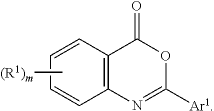

In certain embodiments, Ar¹ is a moiety selected from the group consisting of:

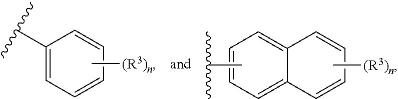

wherein n is a whole number selected from 0-4, 0-3, 0-2, or 0-1;
R³ for each instance is independently alkyl, haloalkyl, perhaloalkyl, alkenyl, alkynyl, cycloalkyl, heterocycloalkyl, aryl, aralkyl, heteroaryl, nitrile, nitro, azido, —OR, —SR, —NR₂, —(C═O)R, —(C═O)OR, —(C═O)NR₂, —(C═NR)NR₂, —(S═O)R, —S(O)₂R, —S(O)₂OR, —S(O)₂NR₂, —(P═O)(OR)₂, or —(CR²₂)ₚY, wherein p for each occurrence is a whole number selected from 1-10; R² for each occurrence is independently selected from the group consisting of hydrogen, alkyl, alkenyl, alkynyl, cycloalkyl, heterocycloalkyl, aryl, and heteroaryl; and Y for each occurrence is selected from the group consisting of OR, SR, NR₂, —(C═O)R, —(C═O)OR, —O(C═O)R, —O(C═O)OR, —(C═O)NR₂, —(NR)(C═O)R, —(NR)(C═O)OR, —O(C═O)NR₂, —O(C═NR)NR₂, —(NR)(C═O)NR₂, —(C═NR)NR₂, —(NR)(C═NR)NR₂, —(S═O)R, —S(O)₂R, —S(O)₂OR, —S(O)₂NR₂, —OS(O)₂R, —(NR)S(O)₂R, —OS(O)₂OR, —OS(O)₂NR₂, —(NR)S(O)₂NR₂, —(NR)S(O)₂OR, and —(P═O)(OR)₂.

In instances in which Ar¹ is

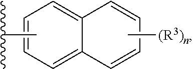

Ar¹ can be selected from a moiety selected from the group consisting of:

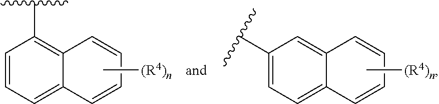

wherein each R³ can be covalently bonded to any position on the napththyl moiety valence permitting.

In certain embodiments, Ar¹ is selected from the group consisting of

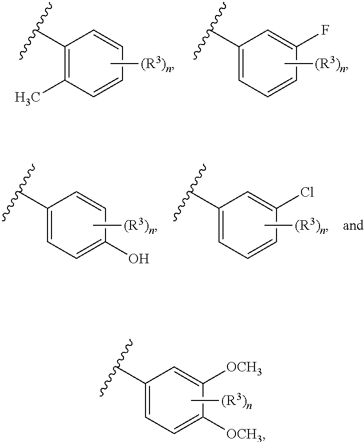

wherein is 0-3 and R³ is as defined in any embodiment described herein.

In certain embodiments, Ar¹ is selected from the group consisting of 4-bromophenyl, 4-hydroxy-phenyl, 2-methyl-phenyl, 3-chloro-phenyl, 4-fluoro-phenyl, 3,4-dimethoxy-phenyl, and 2-naphthyl.

In certain embodiments, R¹ for each instance is independently alkyl, haloalkyl, perhaloalkyl, cycloalkyl, heterocycloalkyl, aryl, aralkyl, heteroaryl, nitrile, nitro, —OR, —SR, —NR₂, —(C═O)NR₂, —(NR)(C═O)R, or —(NR)(C═O)OR. In certain embodiments, at least one R¹ is nitro. In certain embodiments, m is 1 and R¹ is nitro.

In certain embodiments, R² for each occurrence is independently selected from the group consisting of hydrogen and alkyl. In certain embodiments, R² is hydrogen.

In certain embodiments, R³ for each instance is independently alkyl, haloalkyl, perhaloalkyl, cycloalkyl, heterocycloalkyl, aryl, aralkyl, heteroaryl, nitrile, nitro, —OR, —SR, —NR₂, —(C═O)NR₂, —(NR)(C═O)R, or —(NR)(C═O)OR. In certain embodiments, R³ for each instance is independently alkyl, halide, or —OR. In certain embodiments, R³ for each instance is independently methyl, fluoride, chloride, hydroxy, or methoxy.

R for each instance can independently be selected from the group consisting of hydrogen, alkyl, cycloalkyl, heterocycloalkyl, aryl, heteroaryl, and aralkyl; or two instances of R together with the atom they are covalently bonded form a 3-6 membered cycloalkyl or heterocyloalkyl.

In certain embodiments, the compound is selected from the group consisting of:

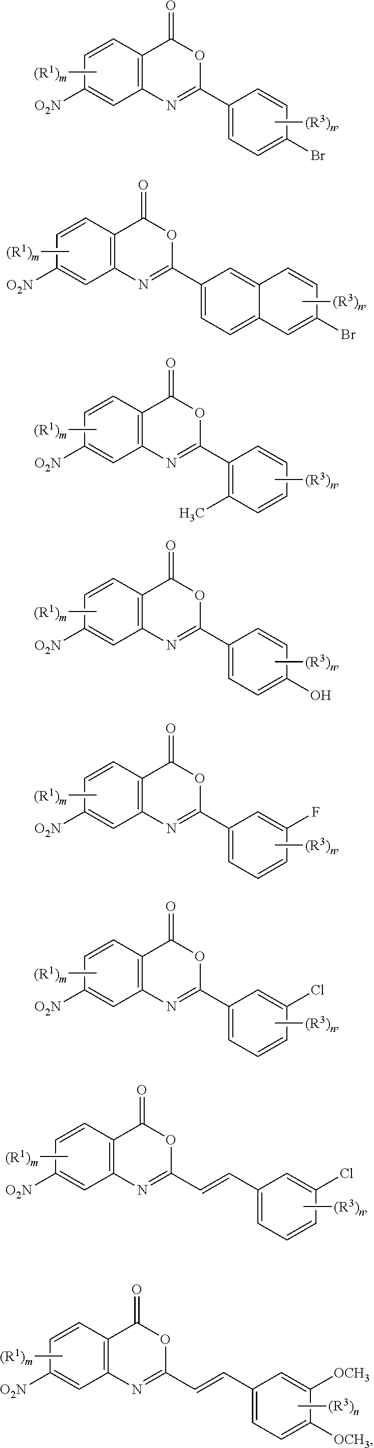

and pharmaceutically acceptable salts thereof, wherein m, n, R¹, and R² are each independently as defined in any embodiment or combination of embodiments described herein.

In certain embodiments, the compound is selected from the group consisting of:

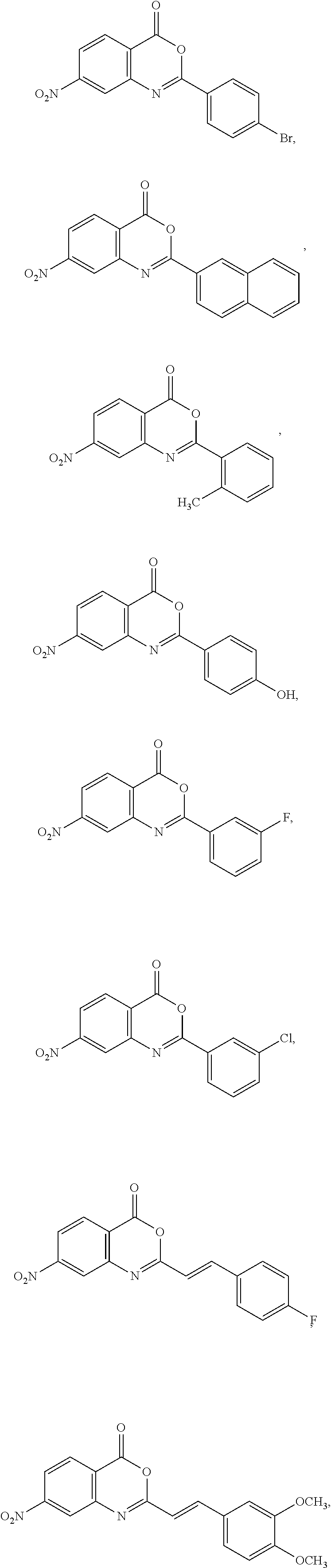

and pharmaceutically acceptable salts thereof.

The compounds described herein can be administered in amount sufficient to reduce the expression of one or more of c-Myc, ESR1, EGFR, HSP90AA1, CCND1, ERBB2, and Bcl-2.

In certain embodiments, the HCC overexpress one or more of c-Myc, ESR1, EGFR, HSP90AA1, CCND1, ERBB2, and Bcl-2. In certain embodiments, the HCC overexpresses Bcl-2.

The method can further comprise determining that one or more of c-Myc, ESR1, EGFR, HSP90AA1, CCND1, ERBB2, and Bcl-2 is overexpressed in the HCC prior to the step of administering the compound.

Specific routes of administration and the dosage regimen will be determined by skilled clinicians, based on factors such as the exact nature of the condition being treated, the severity of the condition, and the age and general physical condition of the patient. In certain embodiments, the compound is administered to the subject orally or intravenously.

Optimal dosages and dosage regimens to be administered may be readily determined by those skilled in the art, and will vary with the mode of administration, the strength of the preparation and the advancement of the disease condition. In addition, factors associated with the particular patient being treated, including patient's sex, age, weight, diet, physical activity, time of administration and concomitant diseases, will result in the need to adjust dosages and/or regimens. In certain embodiments, the compounds described herein are administered to the subject on a once daily, once weekly, twice weekly, thrice weekly, once monthly, or twice monthly basis.

While the dosage will vary depending on the subject's age, body weight, symptom to be treated, desired therapeutic effect, administration route, term of treatment and the like, satisfactory effects can be obtained with the dosage of 0.001-1,000 mg/kg administered systemically in 1 to 5 divided doses a day or as a sustained form.

In this study, a series of benzoxazinone derivatives, designated as ZAK-I-55, ZAK-I-57, ZAK-I-64, ZAK-I-68, ZAK-I-87, ZAK-I-90, ZAK-I-93, and ZAK-I-97, were synthesized via the reaction of substituted 2-aminobenzoic acids (1) with substituted benzoyl chlorides (2 and 3) (FIG. 9). The synthesized compounds were characterized using FT-IR, ¹H-NMR, ¹³C-NMR, and mass spectrometry. The FT-IR analysis exhibited two strong absorption bands in the ranges 1740-1780 cm⁻¹ and 1619-1665 cm⁻¹. For ZAK-I-93 and ZAK-I-97, strong absorption bands in the ranges 978 cm⁻¹ and 969 cm⁻¹, respectively, were attributed to the out-of-plane bending vibration of the C—H bond in E-ethylene. Notably, the characteristic chemical shift (6) values for the olefinic moiety in ZAK-I-93 and ZAK-I-97 were distinctly observed, and their trans-geometry was confirmed with the presence of two doublets along with large coupling constants (J=16.4 and 16.3 Hz, respectively). The structures of the synthesized benzoxazinones (ZAK-I-55, ZAK-I-57, ZAK-I-64, ZAK-I-68, ZAK-I-87, ZAK-I-90, ZAK-I-93, and ZAK-I-97) were confirmed using ¹H-NMR and ¹³C NMR analysis.

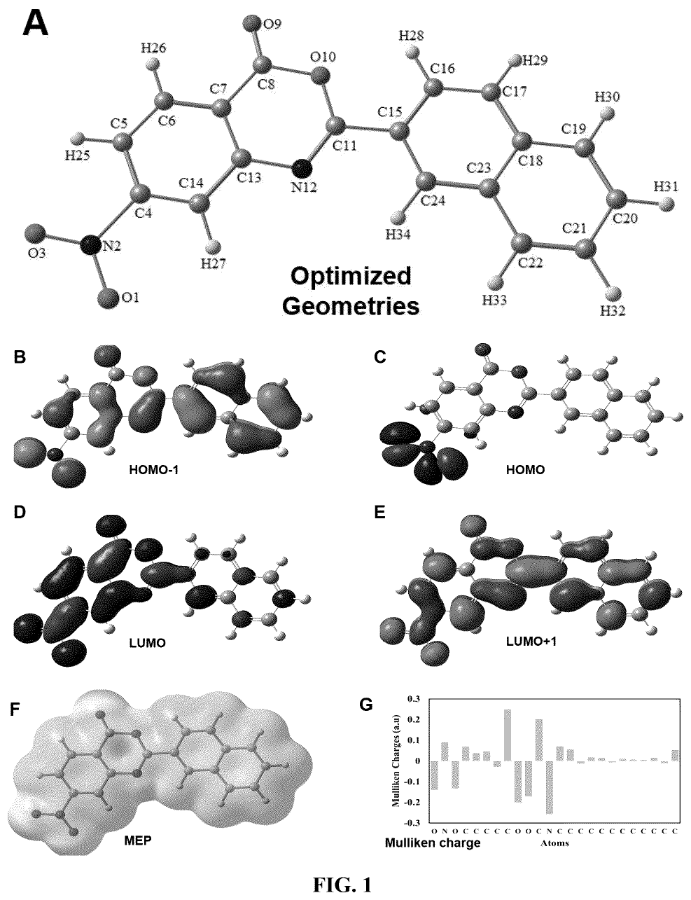

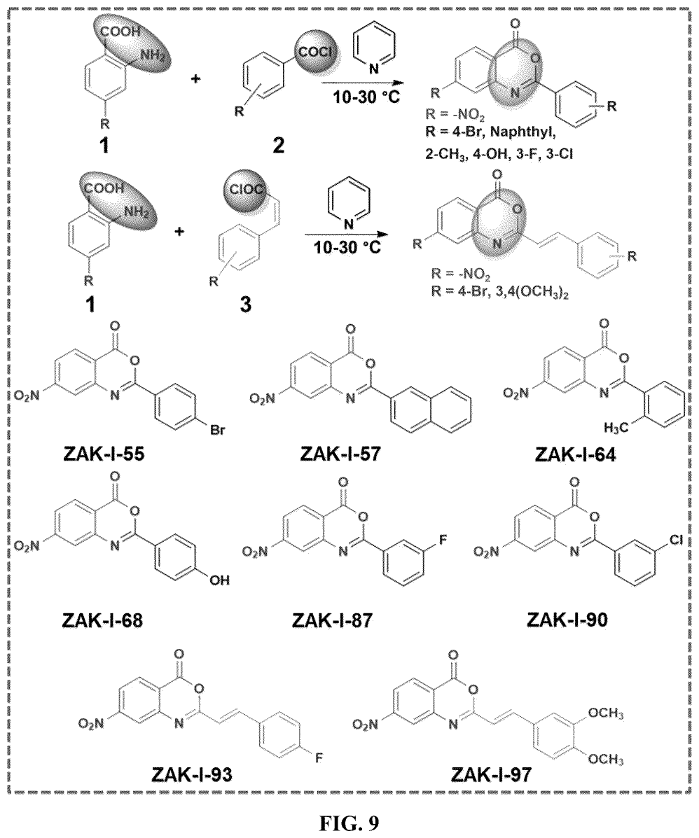

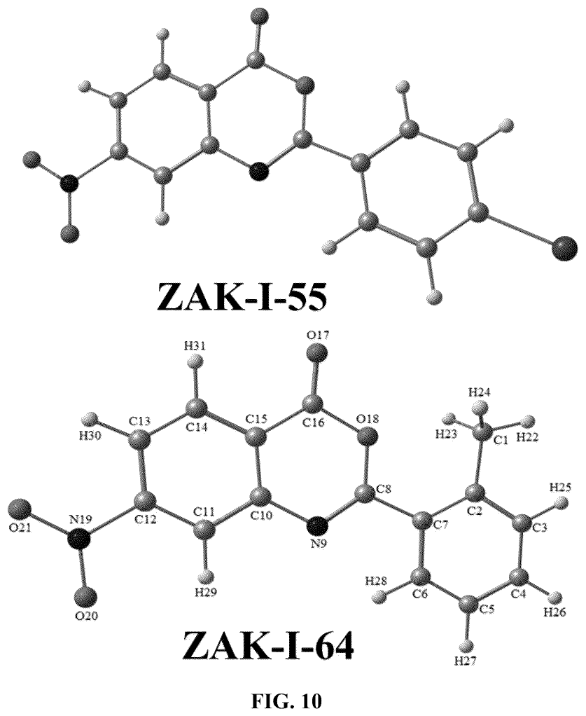

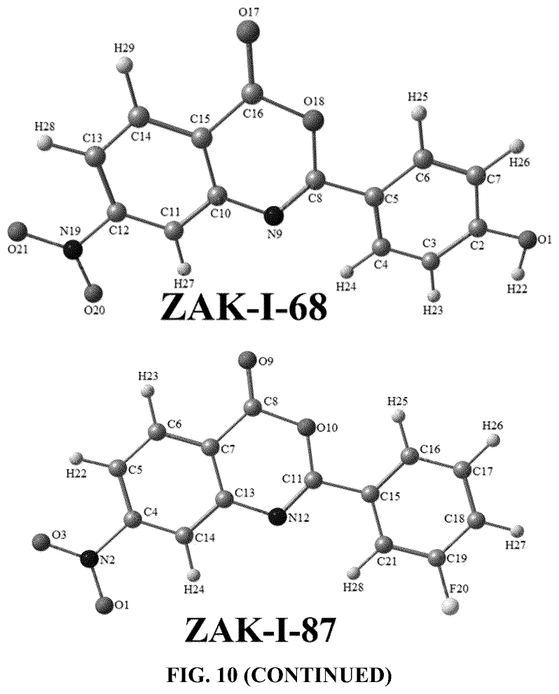

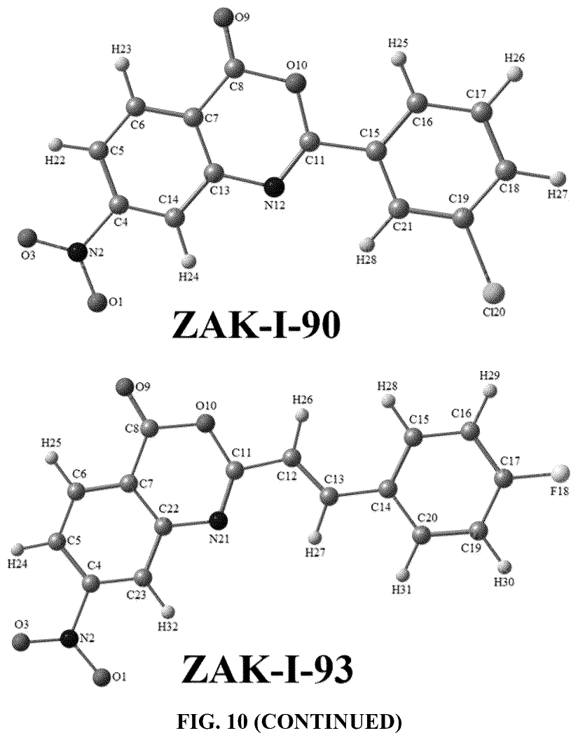

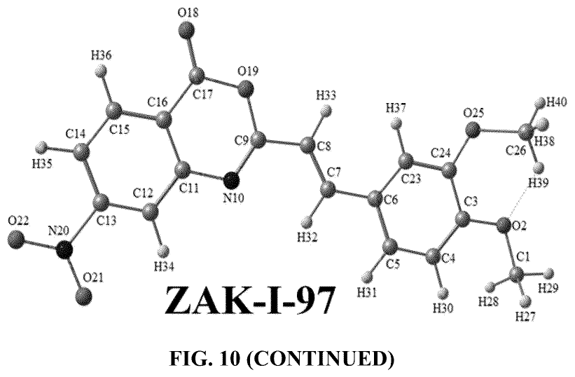

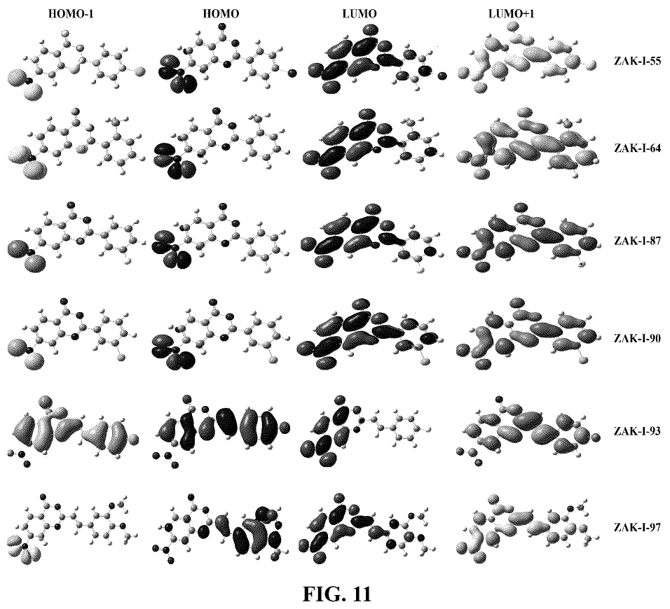

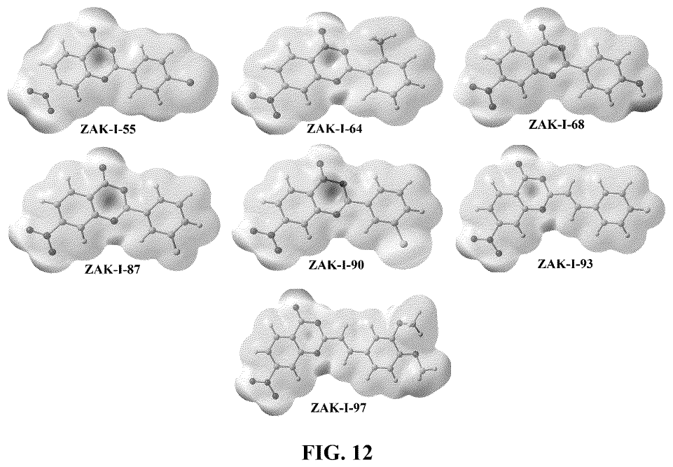

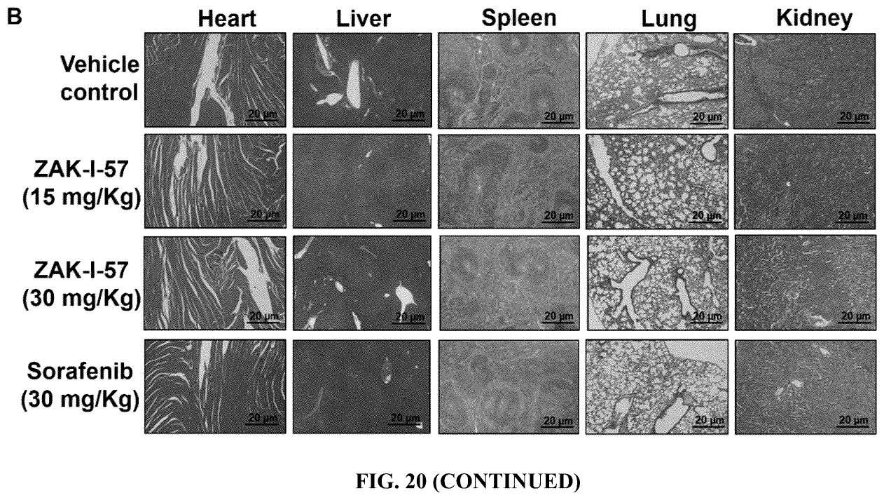

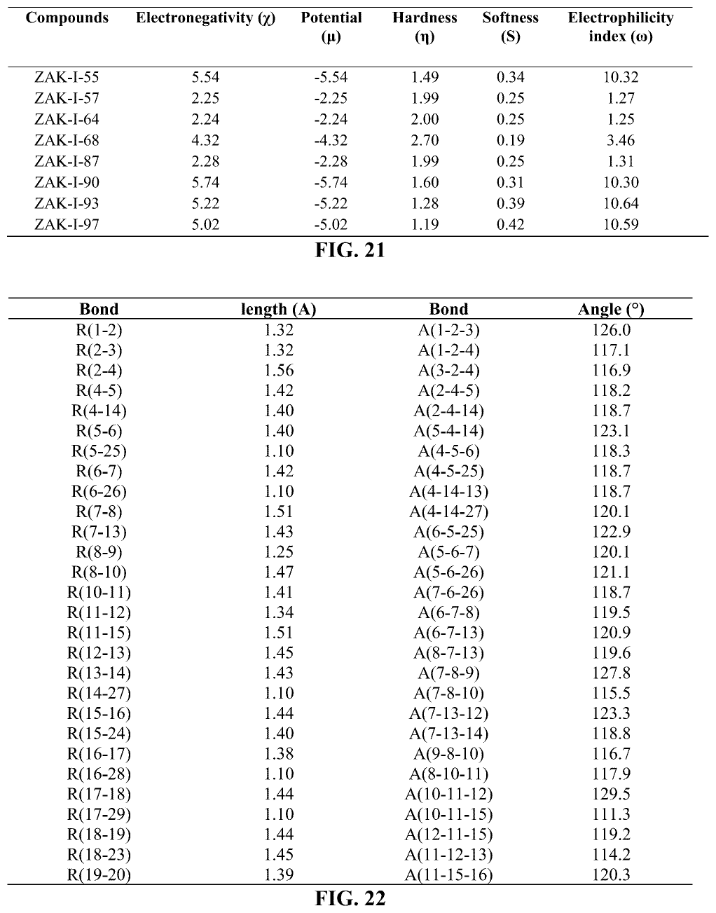

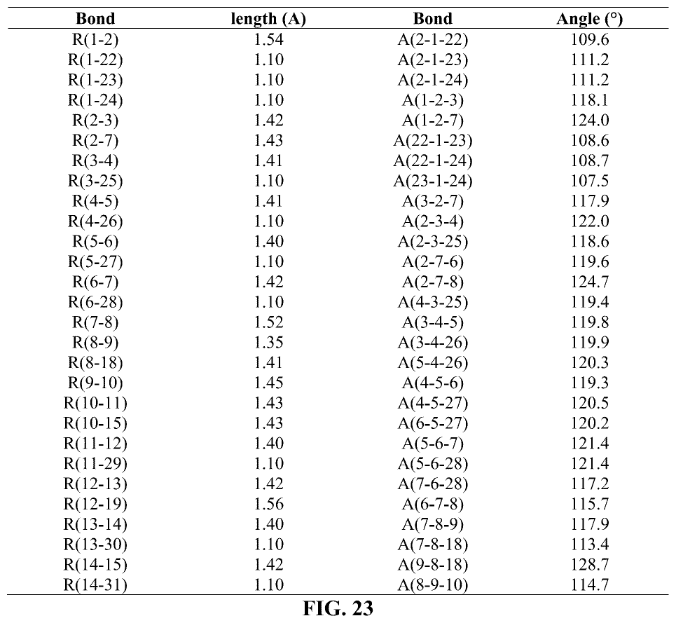

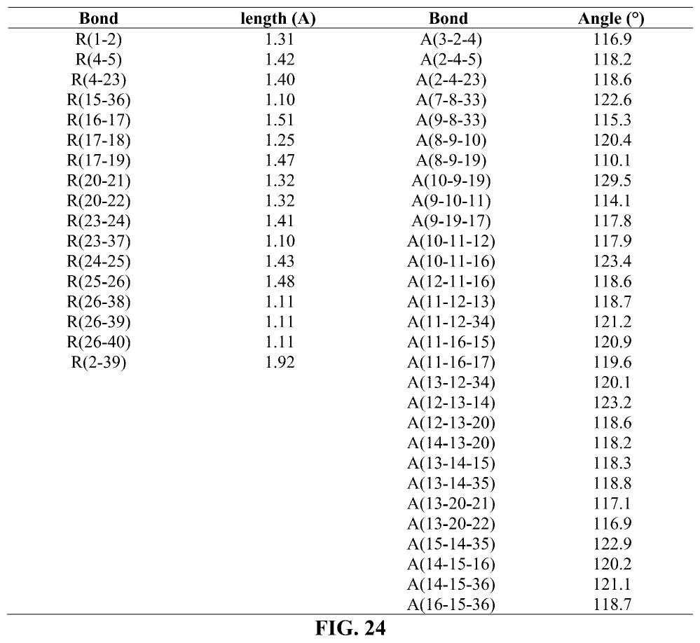

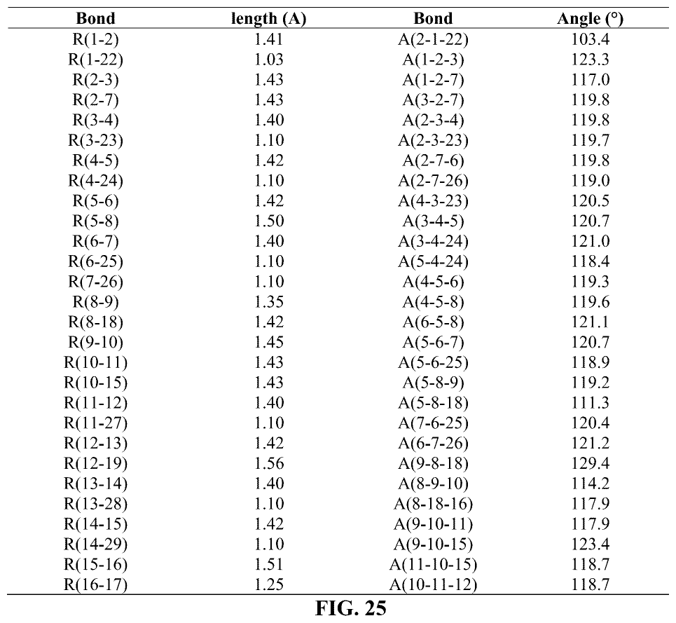

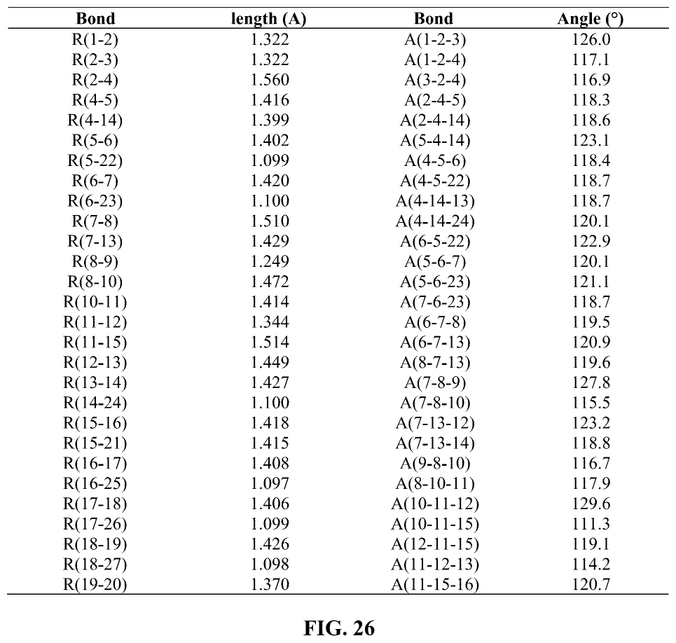

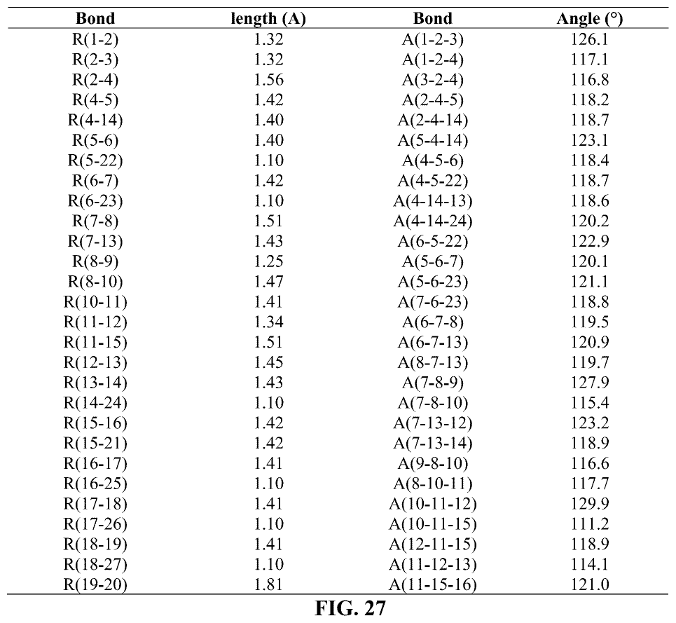

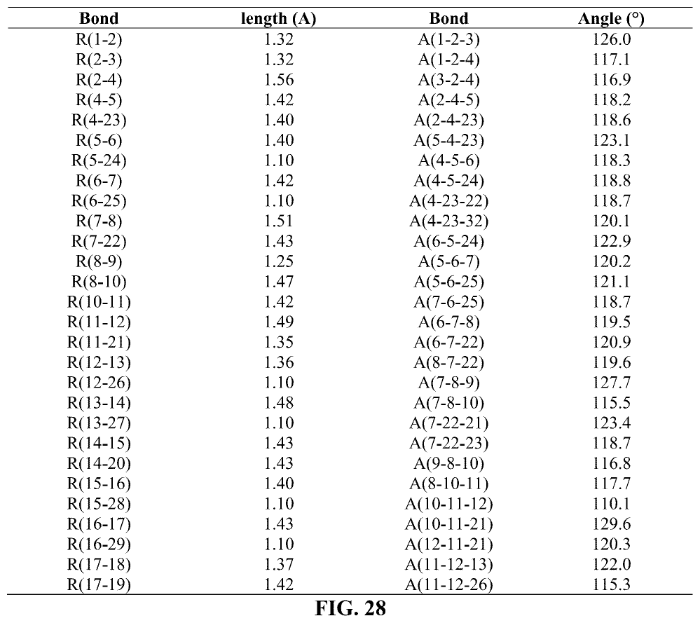

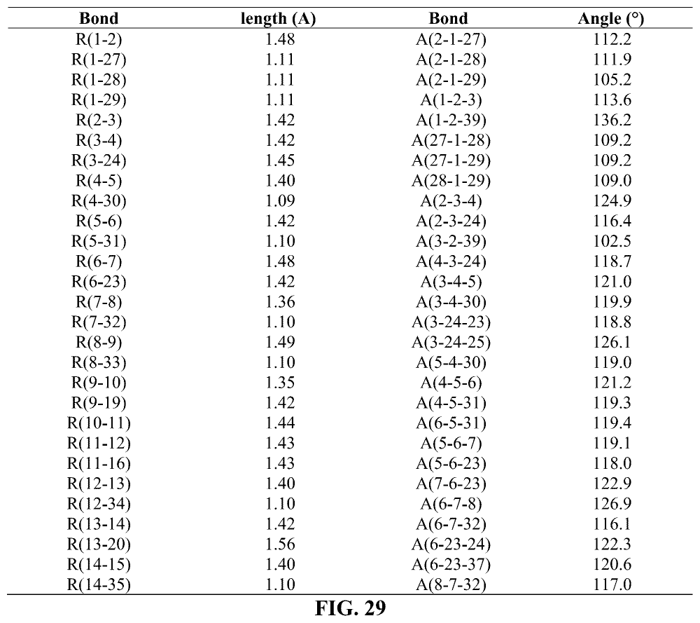

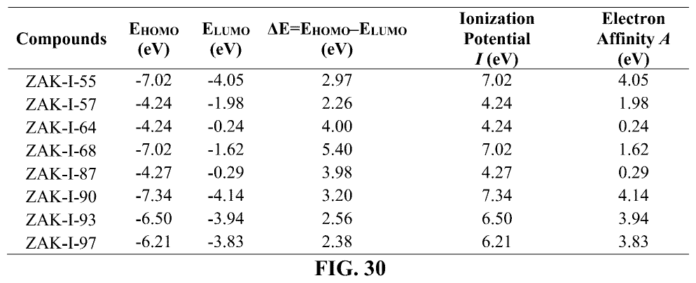

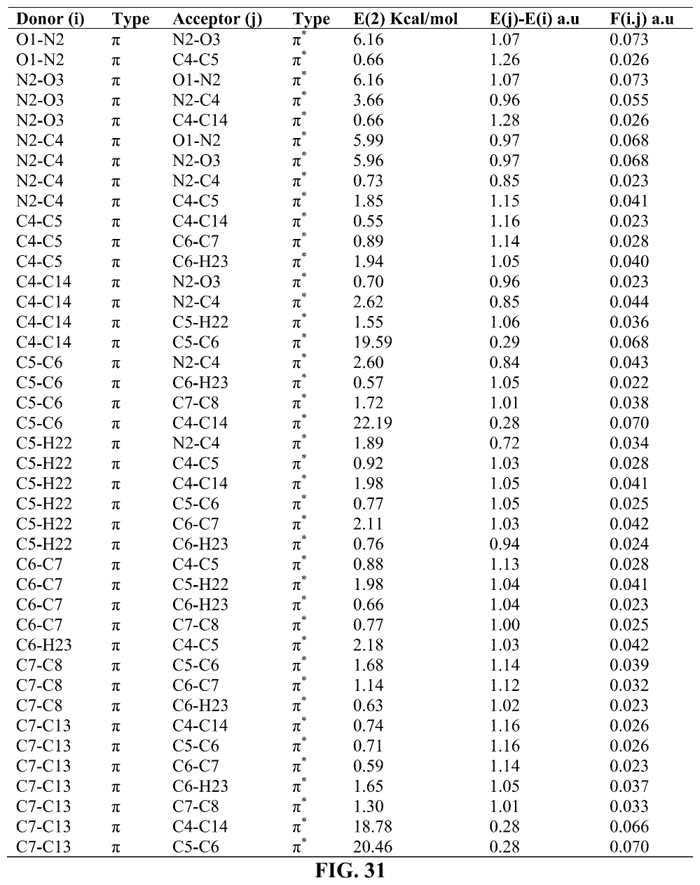

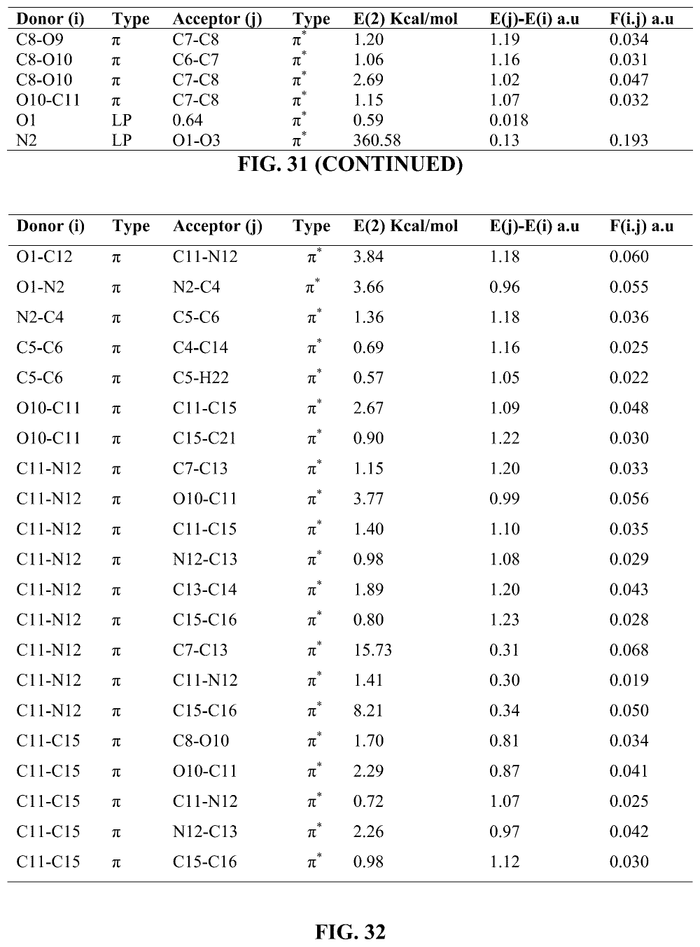

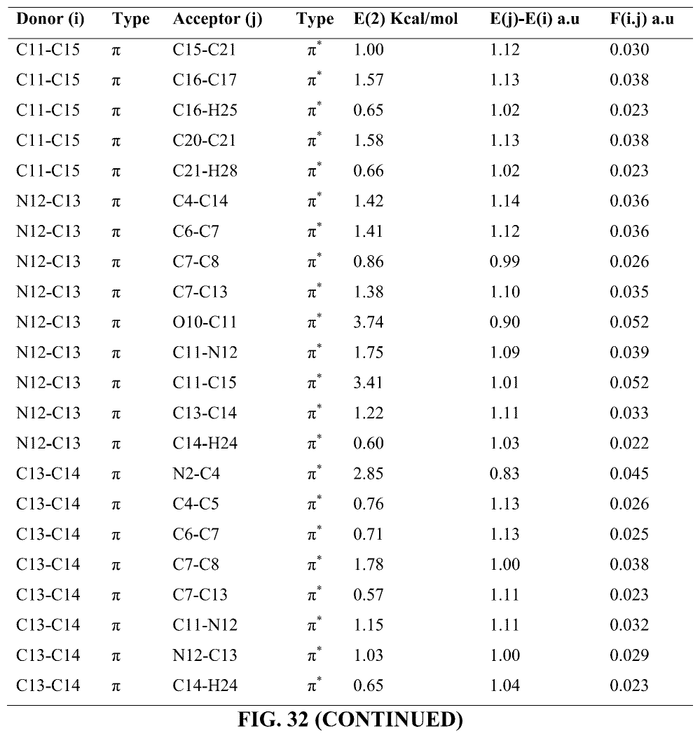

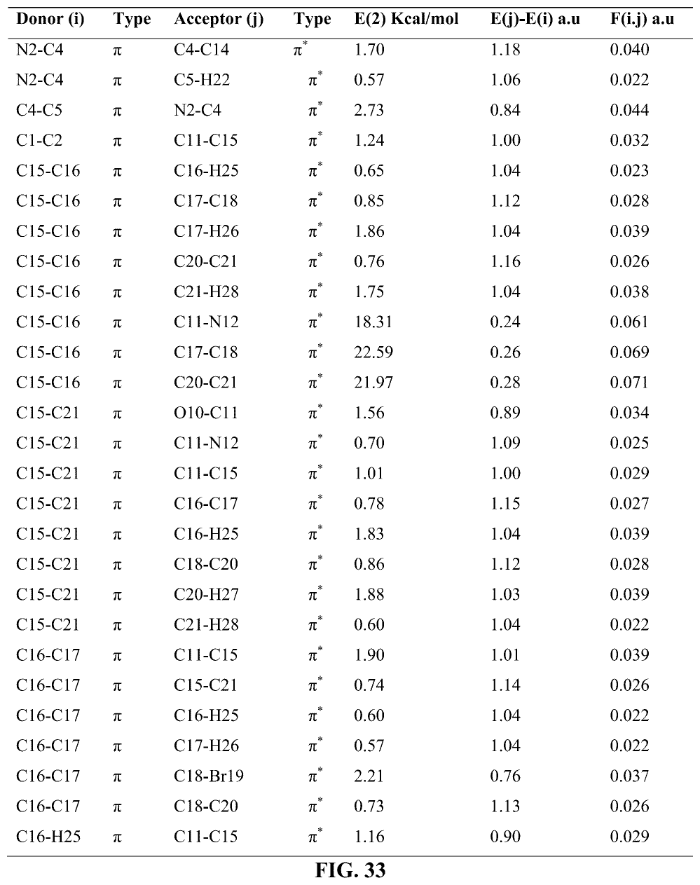

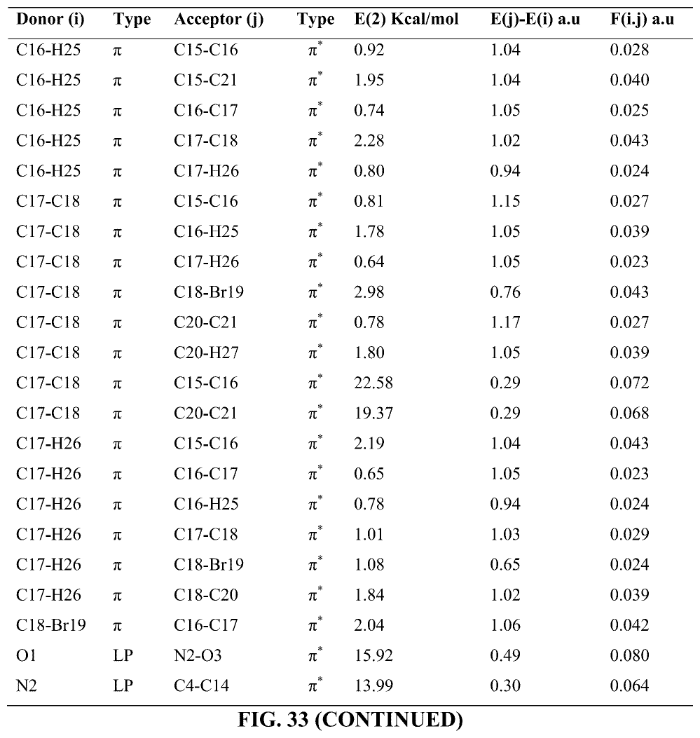

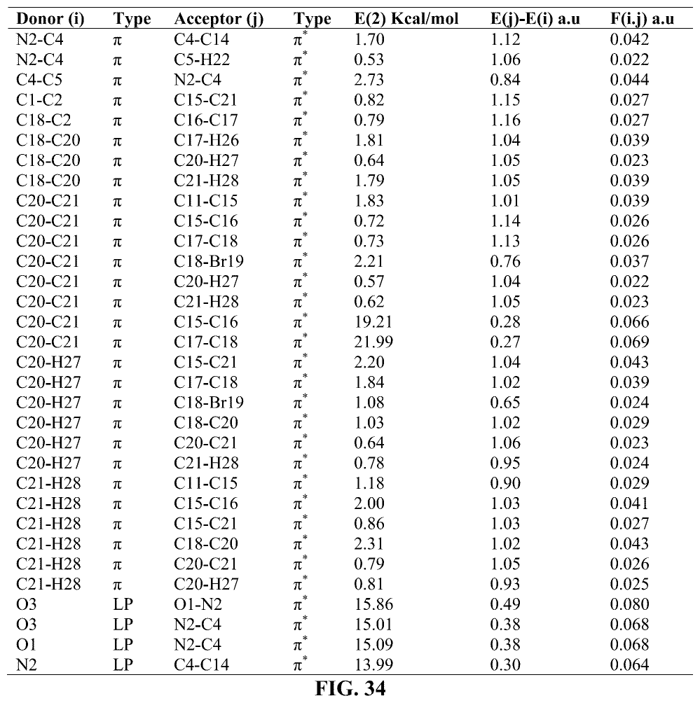

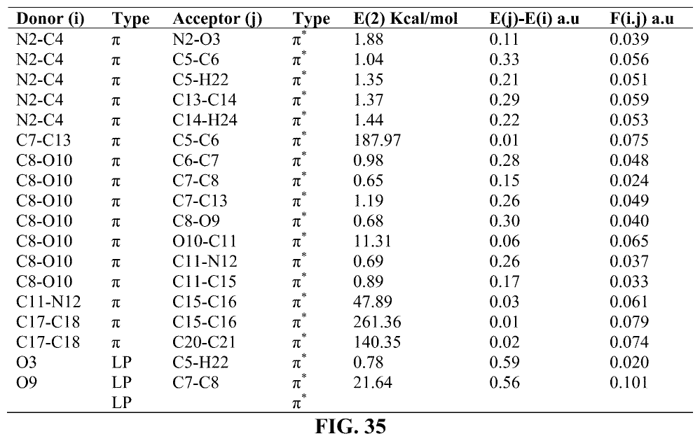

Comprehensive DFT analysis of benzoxazinone derivatives further provides critical insights into their geometric, electronic, and reactivity characteristics, which are essential for their potential applications in biological systems. The global reactivity indices analysis (FIG. 21) identifies ZAK-I-97 and ZAK-I-93 as highly electrophilic and chemically soft, making them well-suited for electron-rich environments. In contrast, ZAK-I-57 and ZAK-I-64 have emerged as potent electron donors. The optimized geometries highlighted the influence of various substituents on the benzoxazinone core (FIGS. 22-29). For instance, the incorporation of a naphthyl group in ZAK-I-57 and a hydroxyl group in ZAK-I-68 introduced significant electronic effects, which likely enhanced the overall reactivity of these molecules (FIGS. 1A and 10). Frontier Molecular Orbital (FMO) analysis revealed that ZAK-I-57 exhibited the smallest HOMO-LUMO gap (ΔE=2.26 eV) (FIGS. 1B-IE, 11, and 30), indicating high reactivity and a propensity for electron transfer processes. These properties are crucial for facilitating interactions with biological targets, such as charge transfer or redox modulation in anticancer applications. The Natural Bond Orbital (NBO) analysis further emphasizes the extensive π-π* interactions in ZAK-I-87, with particularly high stabilization energies, reflecting robust electron delocalization and contributing to its structural stability (FIGS. 31-35). Molecular electrostatic potential (MEP) mapping (FIGS. 1F and 12) and Mulliken charge analysis (FIGS. 1G and 13) provided additional layers of understanding, elucidating the electrostatic interactions and charge distributions that define the reactivity patterns of these derivatives. Taken together, the DFT study provides theoretical support for the potential of ZAK-I-57 and ZAK-I-87 as candidates for further biological evaluation, particularly in the context of anticancer applications such as HCC.

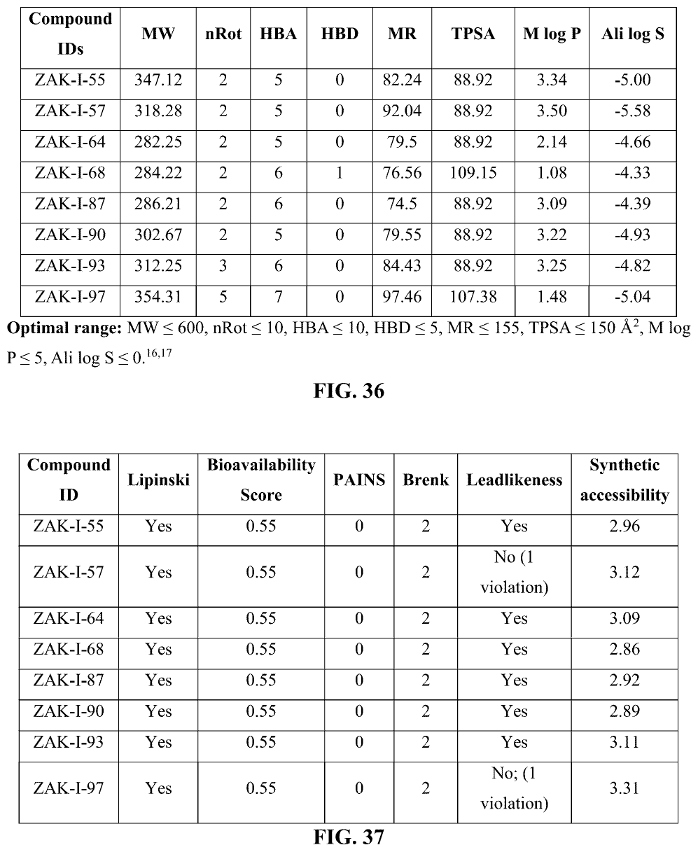

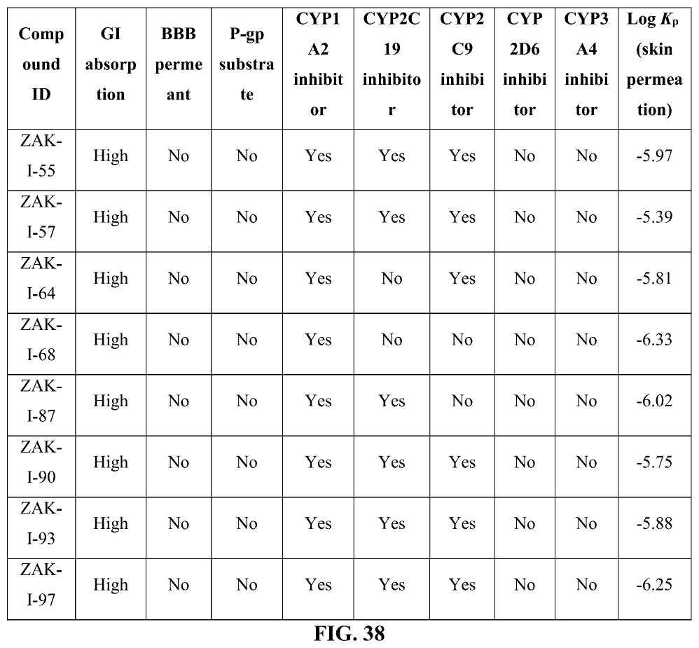

The physicochemical, pharmacokinetic, drug-likeness, and medicinal chemistry properties of benzoxazinone derivatives were predicted using SwissADME (FIGS. 36-38). All compounds adhered to Lipinski's rule of five, exhibited high GI absorption, and showed no BBB permeability. Notably, most derivatives inhibited CYP1A2 and CYP2C9, with variations in CYP2C19 inhibition. Synthetic accessibility scores (2.86-3.31) indicate ease of synthesis, and no PAINS alerts were detected. While Brenk alerts suggested potential specificity. Overall, these derivatives demonstrated favorable DMPK and ADMET profiles, supporting their potential for further development.

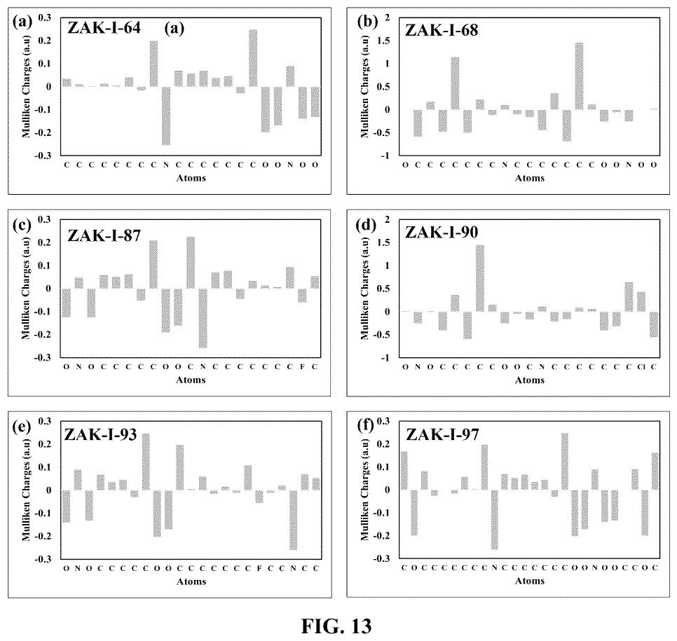

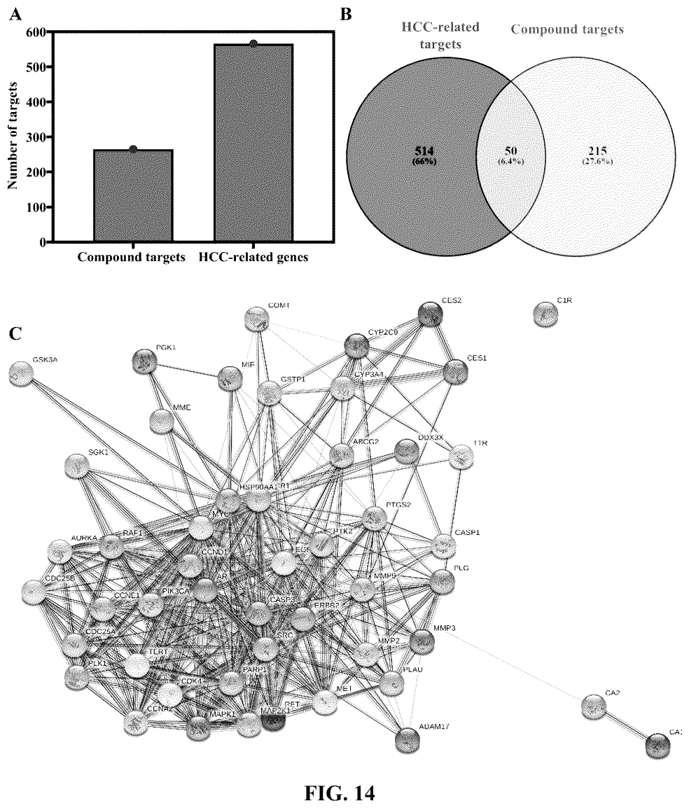

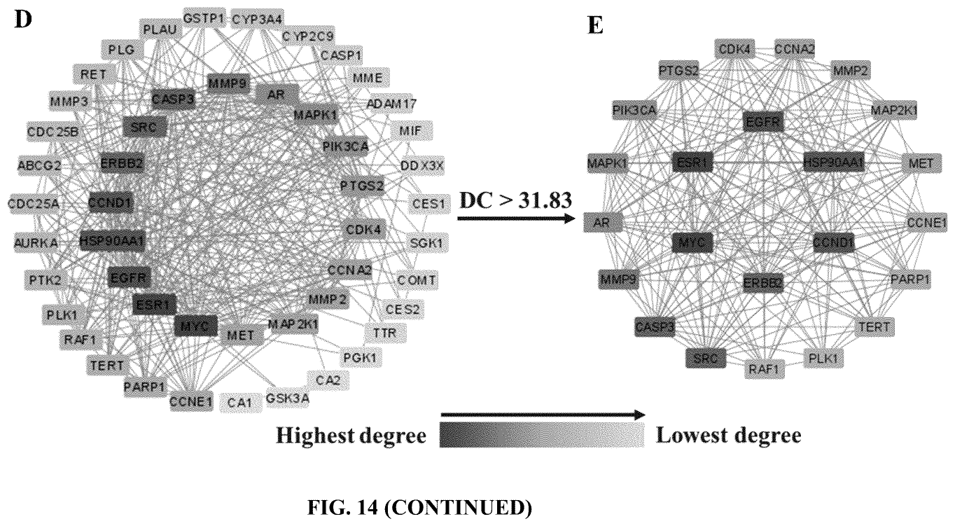

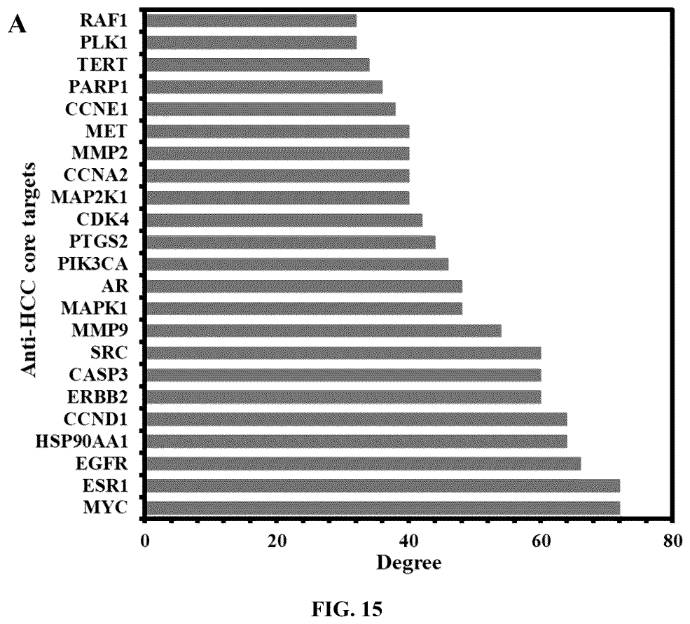

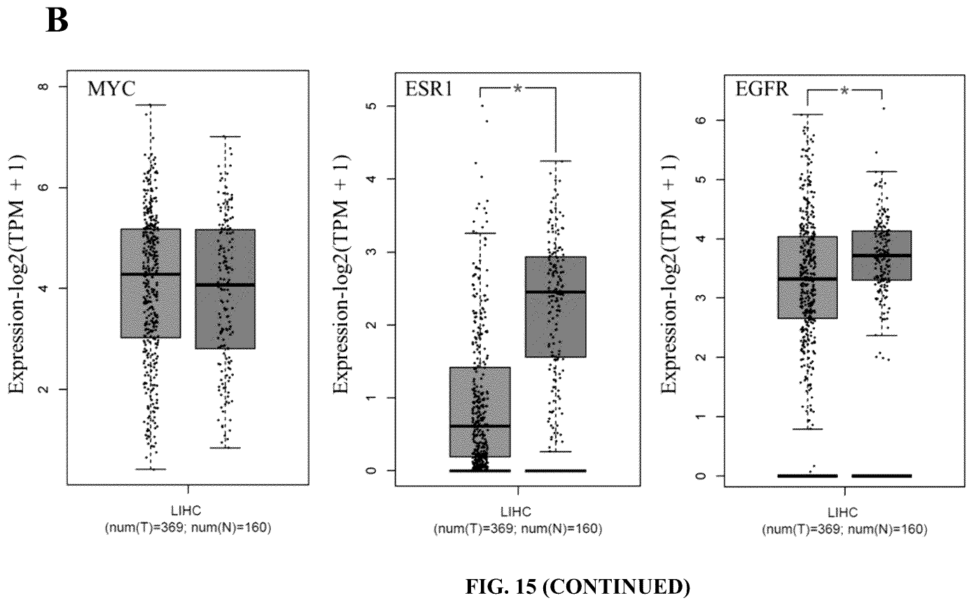

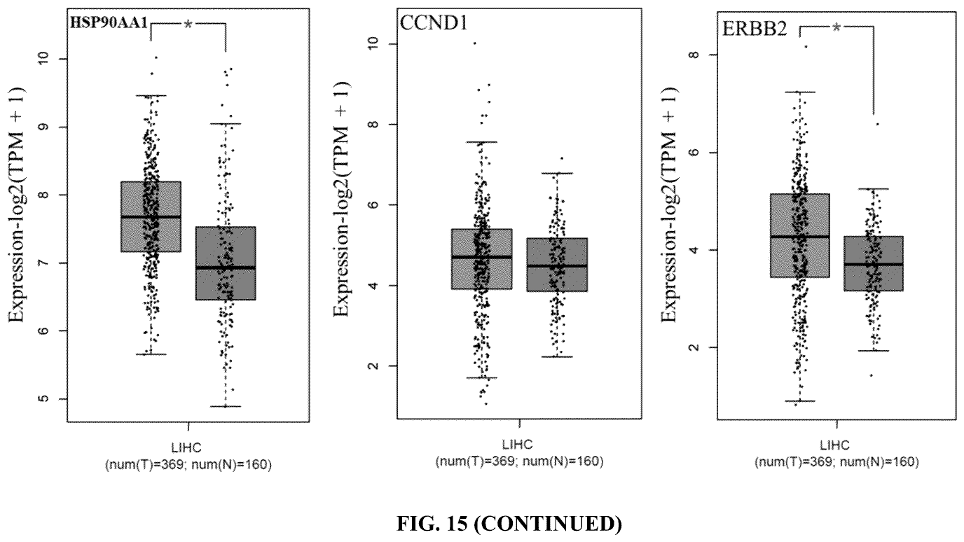

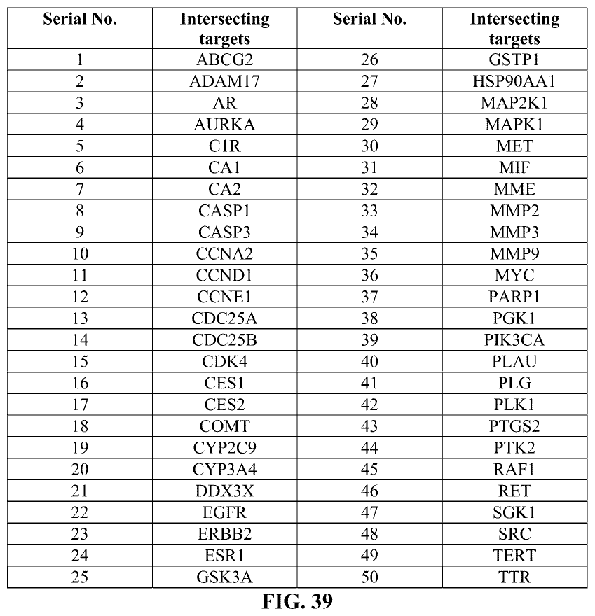

A total of 265 potential protein targets of benzoxazinone derivatives were identified using SwissTargetPrediction (FIG. 14A). 564 HCC-related targets were retrieved from OncoDB, HCC, and Liverome databases (FIG. 14A) and 50 intersecting targets were identified using VENNY 2.1.0 (FIGS. 14B and 39). Subsequently, STRING analysis of these intersecting targets revealed a PPI network comprising 50 nodes and 390 edges, with an average node degree of 15.6 (FIG. 14C). The PPI network was further analyzed in Cytoscape software (version 3.9.0) as shown in FIG. 14D, identifying 23 key nodes (DC>31.83) as potential anti-HCC core targets (FIGS. 14E and 15A). The top six anti-HCC core targets (c-Myc, ESR1, EGFR, HSP90AA1, CCND1, and ERBB2) were validated for differential expression in LIHC samples, showing a strong correlation with HCC progression (FIG. 15B). Due to their critical roles in oncogenesis, these targets were selected for molecular docking to evaluate their interactions with benzoxazinone derivatives.

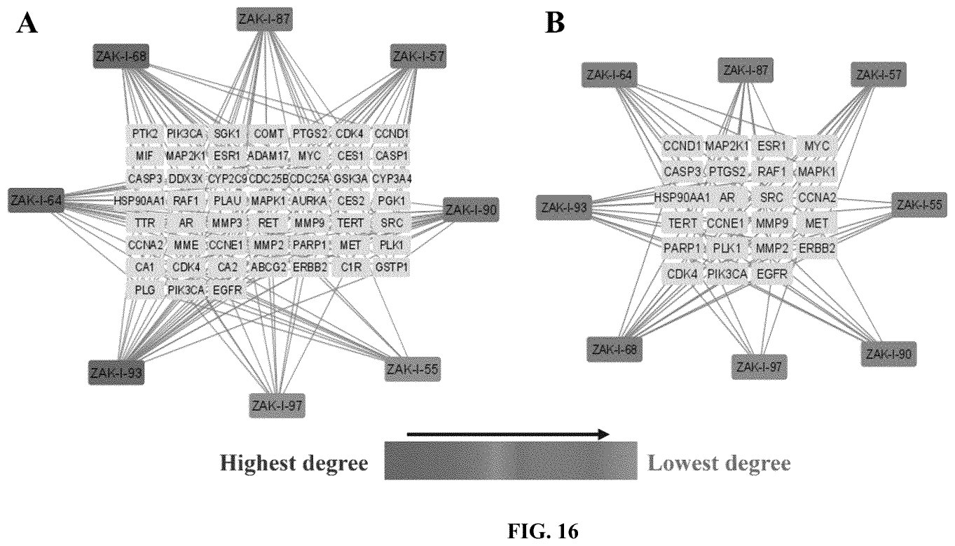

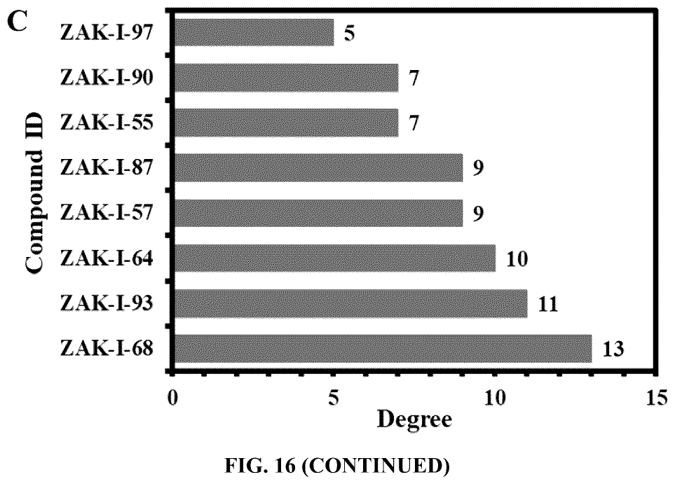

A compound-target network of eight benzoxazinone derivatives with 50 anti-HCC targets was constructed using Cytoscape (FIG. 16A), revealing 60 nodes, 137 edges, four diameters, two radii, 1.199 heterogeneity, 0.077 density, and an average path length of 2.696. Nodes represent target connectivity, while edges indicate compound-target interactions. A hub network was further constructed between eight benzoxazinone derivatives and 23 anti-HCC core targets (FIG. 16B). Degree centrality (DC) analysis identified five key derivatives ZAK-I-57, ZAK-I-64, ZAK-I-68, ZAK-I-87, and ZAK-I-93 exceeding the threshold (average DC>8.875) and interacting with more than eight core targets (FIG. 16C). The hub network confirms the multi-targeting nature of benzoxazinone derivatives, where a single compound modulates multiple anti-HCC targets, and multiple compounds engage the same oncogenic target, suggesting a synergistic inhibitory effect on HCC progression.

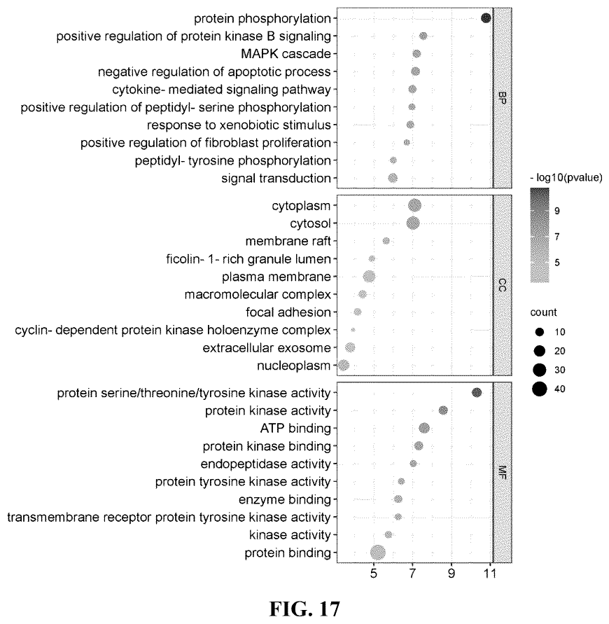

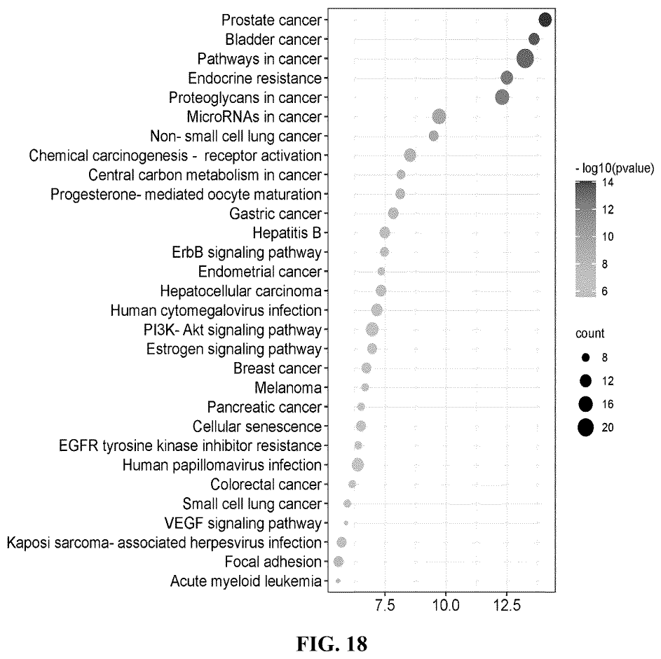

Gene Ontology (GO) enrichment analysis of 50 anti-HCC core targets identified 97 biological processes (BP), 31 cellular components (CC), and 46 molecular functions (MF) (p≤0.05). The top enriched BP terms included protein phosphorylation, negative regulation of the apoptotic process, cytokine-mediated signaling pathway, positive regulation of protein kinase B signaling, and MAPK cascade (FIG. 17). The enriched CC terms localized anti-HCC targets to the cytoplasm, cytosol, plasma membrane, extracellular exosomes, and nucleoplasm, while MF enrichment predominantly involved protein binding, ATP binding, protein serine/threonine/tyrosine kinase activity, protein kinase activity, and protein kinase binding, reinforcing the therapeutic relevance of benzoxazinone derivatives in modulating key molecular functions and cellular processes involved in HCC progression. Kyoto Encyclopedia of Genes and Genomes (KEGG) pathway analysis further elucidated the mechanistic involvement of benzoxazinone derivatives in HCC therapy, identifying six key pathways: cancer-associated signaling (23 targets), proteoglycans in cancer (16), ErbB signaling (9), PI3K-Akt pathway (14), EGFR tyrosine kinase inhibitor resistance (8), and VEGF signaling (7) (FIG. 18). These results highlight the broad involvement of anti-HCC targets in multiple oncogenic pathways, suggesting that benzoxazinone derivatives exert their therapeutic effects through a multi-targeted mechanism, making them promising candidates for HCC treatment.

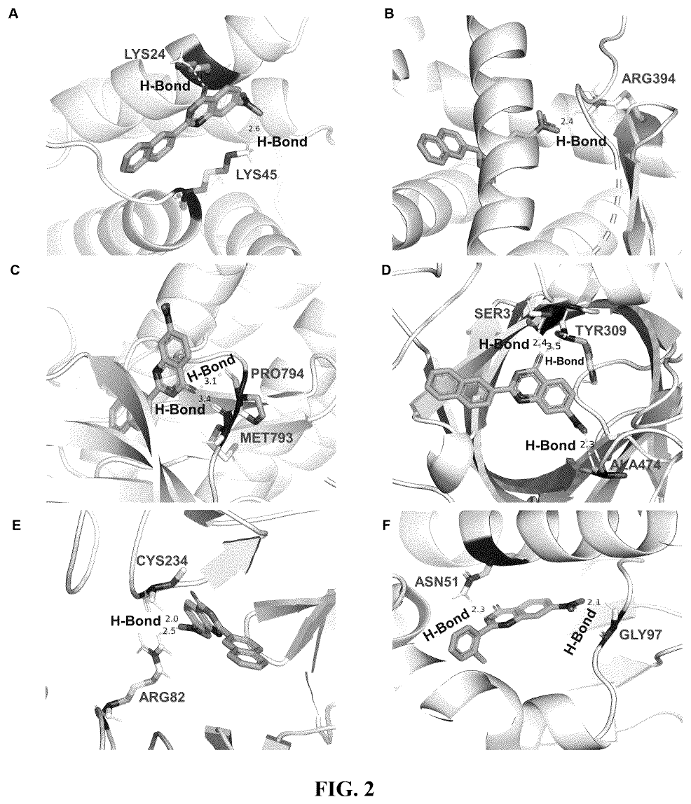

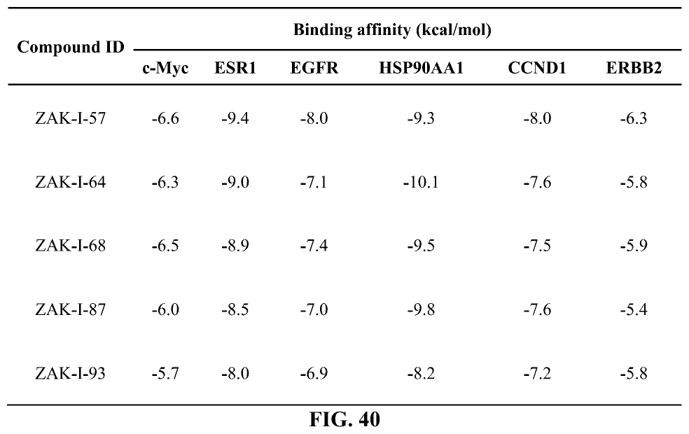

Molecular docking of five key active benzoxazinone derivatives with six anti-HCC core targets (c-Myc, ESR1, EGFR, HSP90AA1, CCND1, and ERBB2) was performed and the binding affinity are summarized in FIG. 40. A lower binding energy signifies stronger ligand-receptor interactions, indicating high binding affinity. Docking results revealed that all benzoxazinone derivatives exhibited strong binding affinities toward the six core targets, supporting their multi-targeted potential in HCC therapy. Among these, ZAK-I-57 (−6.6 kcal/mol) and ZAK-I-68 (−6.5 kcal/mol) showed the strongest binding to c-Myc, while ZAK-I-57 (−9.4 kcal/mol) and ZAK-I-64 (−9.0 kcal/mol) had the highest affinity for ESR1. For EGFR, ZAK-I-57 (−8.0 kcal/mol) and ZAK-I-68 (−7.4 kcal/mol) exhibited the lowest binding energies. ZAK-I-64 (−10.1 kcal/mol) and ZAK-I-87 (−9.8 kcal/mol) showed the strongest binding to HSP90AA1. For CCND1, ZAK-I-57 demonstrated the highest binding affinity with an energy score of −8.0 kcal/mol, while ZAK-I-64 and ZAK-I-87 showed identical binding affinities of −7.6 kcal/mol each. Lastly, ZAK-I-57 (−6.3 kcal/mol) and ZAK-I-68 (−5.9 kcal/mol) exhibited strong affinity for ERBB2. The docked ligand-receptor complexes with the best binding affinities are depicted in FIGS. 2A-F. ZAK-I-57 established hydrogen bonds with c-Myc (LYS24: 2.4 Å, LYS45: 2.6 Å), ESR1 (ARG394: 2.4 Å), EGFR (MET793: 3.4 Å, PRO794: 3.16 Å), CCND1 (TYR309: 3.5 Å, SER311: 2.4 Å, ALA474: 2.3 Å), and ERBB2 (ARG82: 2.5 Å, CYS234: 2.0 Å), engaging critical residues involved in oncogenic signaling and transcriptional regulation. ZAK-I-64 exhibited strong hydrogen bonding interactions with HSP90AA1 (ASN51: 2.3 Å, GLY97: 2.1 Å), targeting its chaperone function essential for protein stability in cancer cells. Thus, the docking results indicate that benzoxazinone derivatives, such as ZAK-I-57, ZAK-I-64, and ZAK-I-68, are the most important and significant compounds that may be effective in suppressing oncogene proteins implicated in HCC.

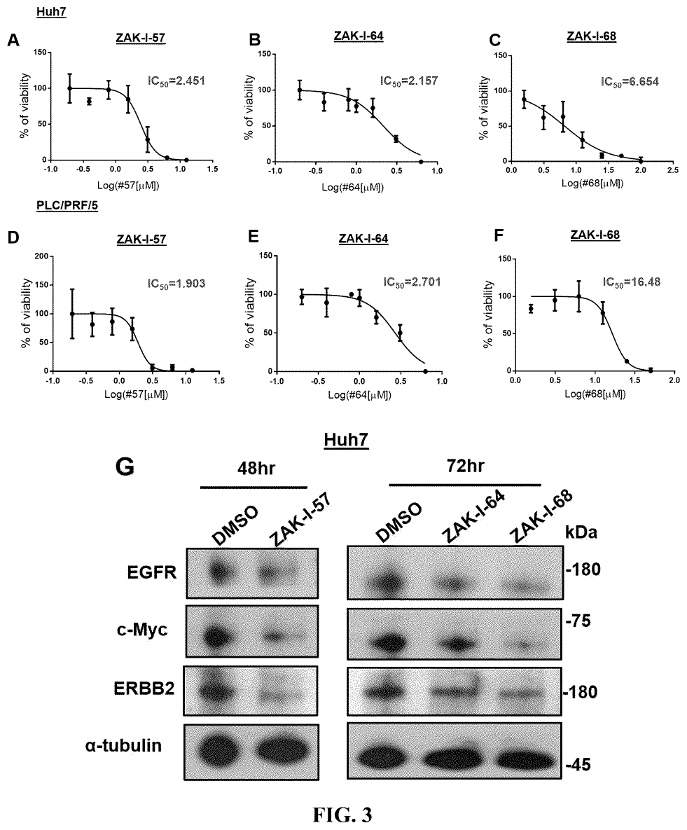

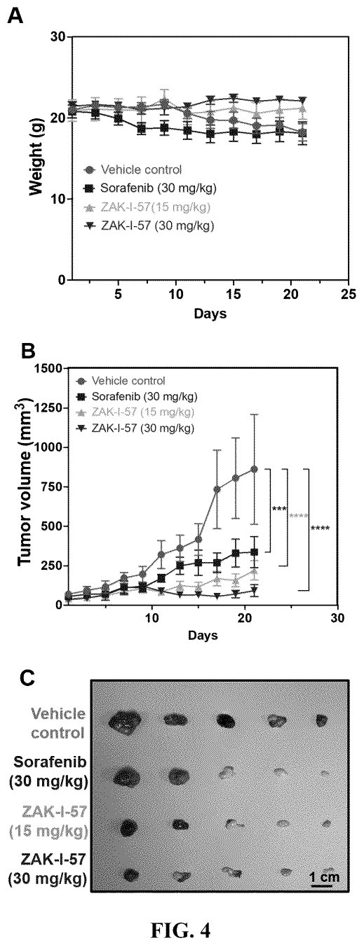

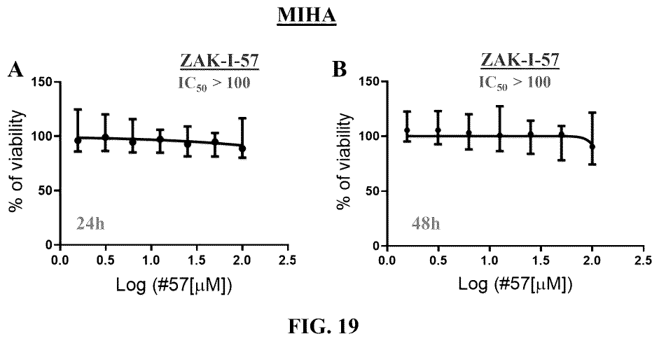

Three benzoxazinone derivatives (ZAK-I-57, ZAK-I-64, and ZAK-I-68) exhibited the most promising results in system pharmacology and molecular docking studies, prompting further investigation of their cytotoxic activity in Huh7 and PLC/PRF/5 cell lines. As shown in FIGS. 3A-F, a concentration-dependent increase in cytotoxicity was observed for all three compounds in both cell lines. Notably, ZAK-I-57 demonstrated the highest toxicity after 24 h of treatment (FIGS. 3A, and 3D), while ZAK-I-64 and ZAK-I-68 exerted comparable cytotoxic effects after 48 h (FIGS. 3B, 3C, 3E, and 3F). Following these promising results, ZAK-I-57 was further evaluated against MIHA, an immortalized normal liver cell line. ZAK-I-57 exhibited negligible cytotoxicity (IC₅₀>100 μM at 24 h and 48 h; FIGS. 19A and 19B), underscoring its selectivity and therapeutic potential. Western blot analysis revealed significant downregulation of EGFR, c-Myc, and ERBB2 after 48 h treatment with ZAK-I-57 (FIG. 3G), whereas ZAK-I-64 and ZAK-I-68 exhibited no appreciable effect at this time point. However, prolonged exposure to 72 h resulted in significant downregulation of these targets by both compounds, with ZAK-I-68 demonstrating greater efficacy than ZAK-I-64. These results, in conjunction with our network pharmacology and molecular docking data, substantiate that ZAK-I-57, ZAK-I-64, and ZAK-I-68 effectively modulate multiple HCC-related targets.

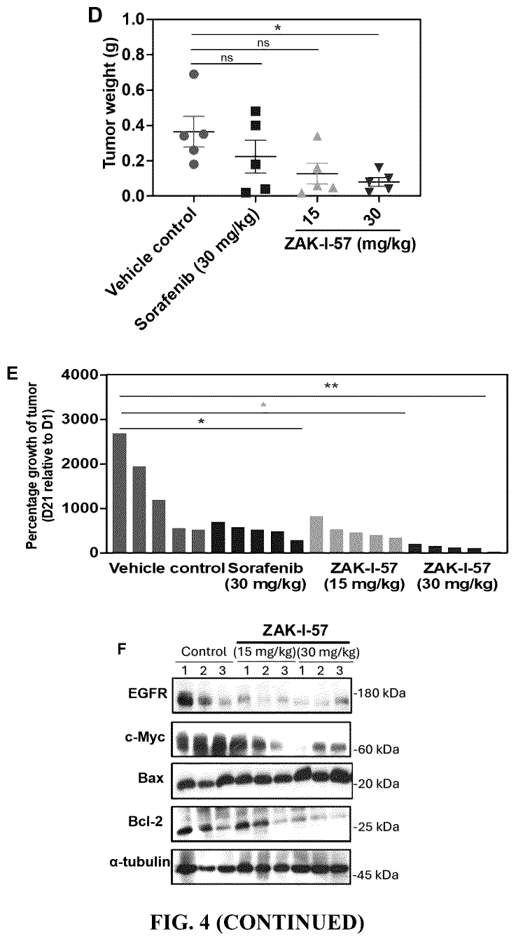

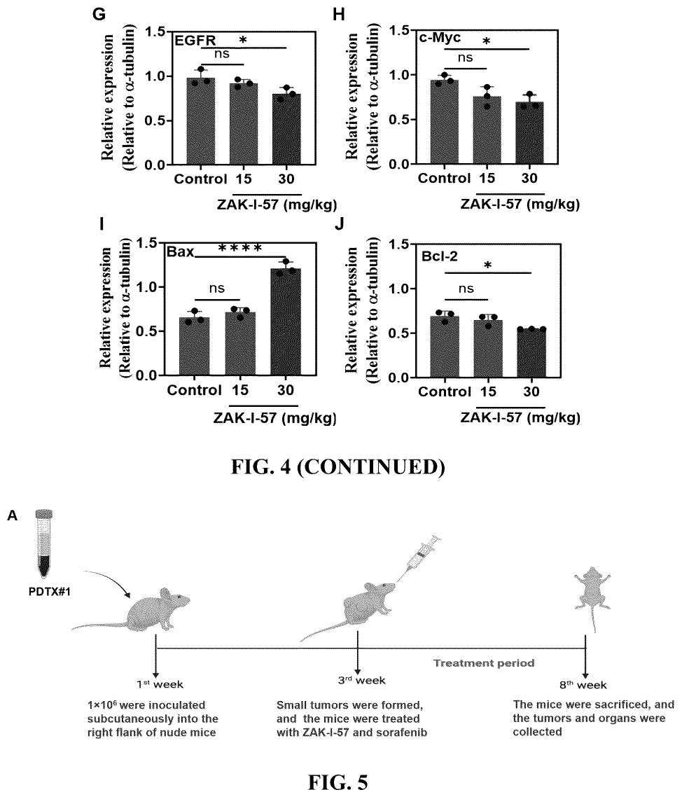

Motivated by its exceptional in vitro performance, we further evaluated the antitumor efficacy of ZAK-I-57 in PLC/PRF/5 tumor-bearing mice and compared it with that of the standard drug, sorafenib. The mice were divided into four groups: vehicle control, sorafenib (30 mg/kg), ZAK-I-57 (15 mg/kg), and ZAK-I-57 (30 mg/kg), with five mice per group. Key metrics, including body weight, tumor volume, and tumor weight, were meticulously measured and assessed throughout the study. The results showed no significant changes (p>0.05) in the body weight of the mice treated with ZAK-I-57 (15 and 30 mg/kg) for 20 days (FIG. 4A). In contrast, the vehicle control and sorafenib groups exhibited only slight variation in body weight. Notably, the groups treated with ZAK-I-57 displayed a marked reduction in tumor volume and weight compared to the vehicle control and sorafenib-treated groups (FIG. 4B-D). Furthermore, the tumor growth percentage results demonstrated that ZAK-I-57 significantly inhibited tumor growth at concentrations of 15 and 30 mg/kg, demonstrating superior efficacy compared to sorafenib at 30 mg/kg (FIG. 4E). Western blot analysis showed that ZAK-I-57 at 30 mg/kg significantly attenuated the expression of key oncogenic proteins, including EGFR, c-Myc, and Bcl-2, compared to the control group (FIG. 4 F-J). Notably, this concentration also induced an increase in the expression of Bax, a pro-apoptotic protein, indicating a dual mechanism of action of the compound. Hence, the significant downregulation of oncogenic proteins and upregulation of pro-apoptotic proteins underscores ZAK-I-57's multifaceted approach to inhibiting HCC progression, making it a promising therapeutic candidate.

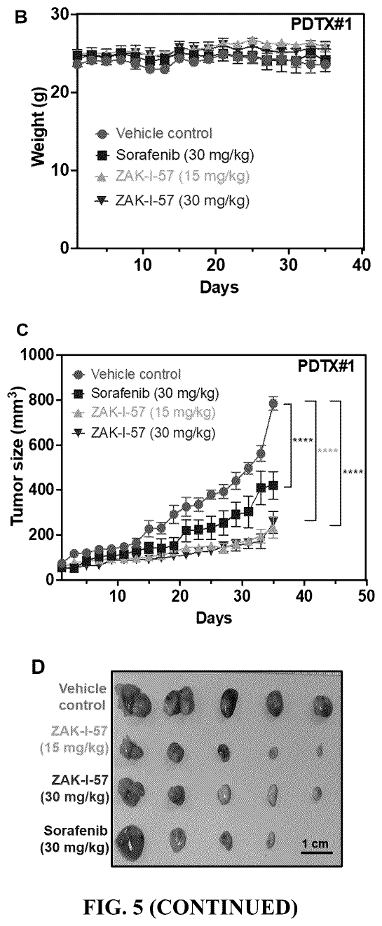

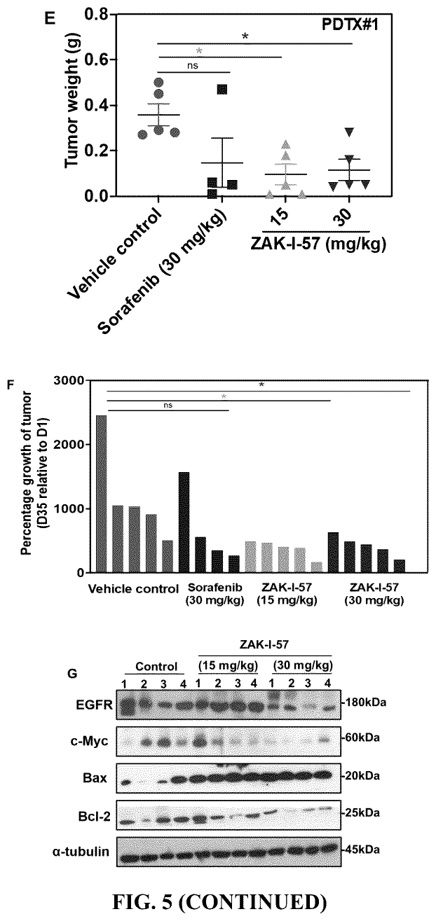

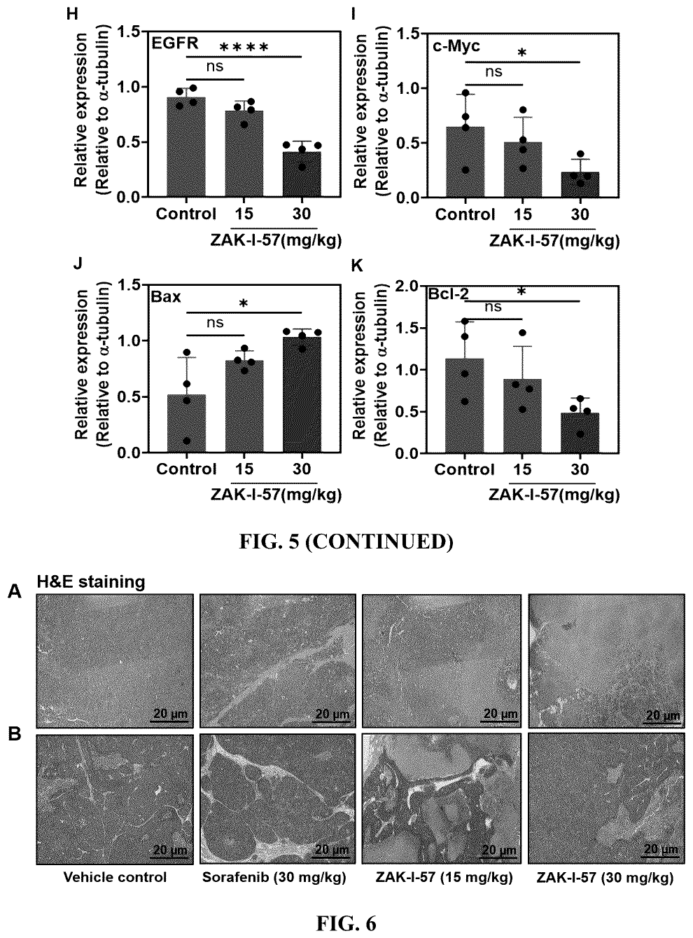

We extended our investigation to evaluate its potential in the PDTX (PDTX #1) derived HCC model (FIG. 5A), comparing it to the standard drug sorafenib. Mice were divided with each group comprising five mice, except for the sorafenib group, which consisted of four mice. Notably, no significant difference (p>0.05) was observed between the ZAK-I-57 treatment groups and control group, indicating a lack of significant toxicity. Slight variations in the weight of the vehicle control and sorafenib groups were also observed compared to the ZAK-I-57 treated groups at both dosages (FIG. 5B). FIGS. 5C and 5D demonstrate that ZAK-I-57 significantly inhibited tumor progression in a dose-dependent manner, with a significant reduction in tumor weight at the dose of 30 mg/kg (FIG. 5E). FIG. 5F illustrates that the percentage of tumor growth was markedly diminished in the groups treated with ZAK-I-57 (15 mg/kg and 30 mg/kg) compared to that in the vehicle control group. Western blot analysis presented in FIGS. 5G-K further demonstrate that at a dose of 30 mg/kg, ZAK-I-57 significantly (p<0.05) downregulated EGFR and c-Myc, two pivotal oncogenic drivers in HCC, indicating strong inhibition of proliferative signaling pathways. Simultaneously, ZAK-I-57 demonstrated a potent pro-apoptotic effect by significantly upregulating Bax and downregulating Bcl-2 at 30 mg/kg dose. This dual modulation not only curtails tumor growth but actively promotes tumor cell death, showcasing ZAK-I-57's comprehensive anti-tumor capabilities.

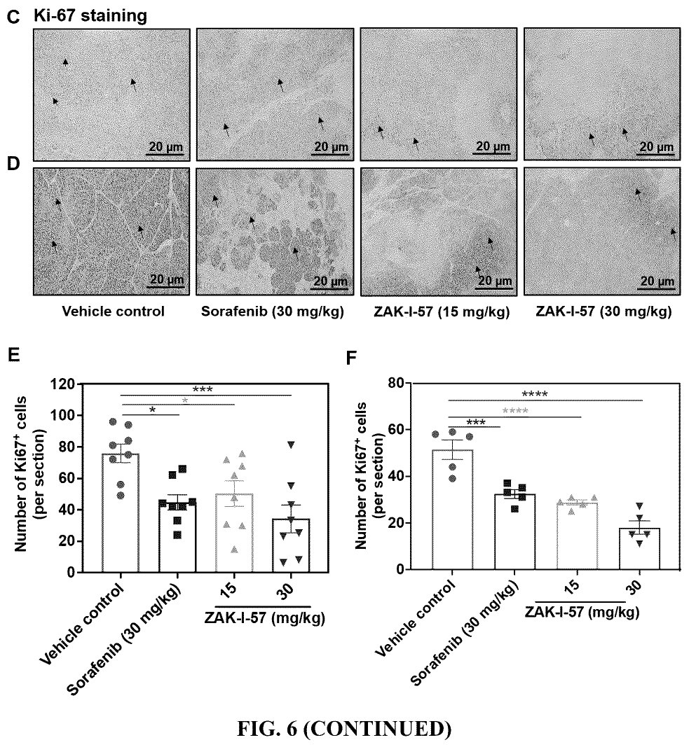

Histopathological and proliferation analysis using H&E and Ki-67 staining confirmed the significant antitumor effects of ZAK-I-57 in PLC/PRF/5 tumor-bearing and PDTX mouse model tissues, indicating its potential as a superior therapeutic agent compared to sorafenib, as shown in FIGS. 6A-F. H&E staining demonstrated a marked reduction in tumor cellularity following treatment with ZAK-I-57 (15 and 30 mg/kg) and sorafenib (30 mg/kg) compared to the vehicle control. Notably, the 30 mg/kg dose of ZAK-I-57 induced substantial decrease in cell density and necrosis, paralleling the effects observed with sorafenib. In addition, ZAK-I-57 exhibited antiproliferative activity, as evidenced by the inhibition of Ki-67 expression in both HCC xenograft models (FIGS. 6E-F).

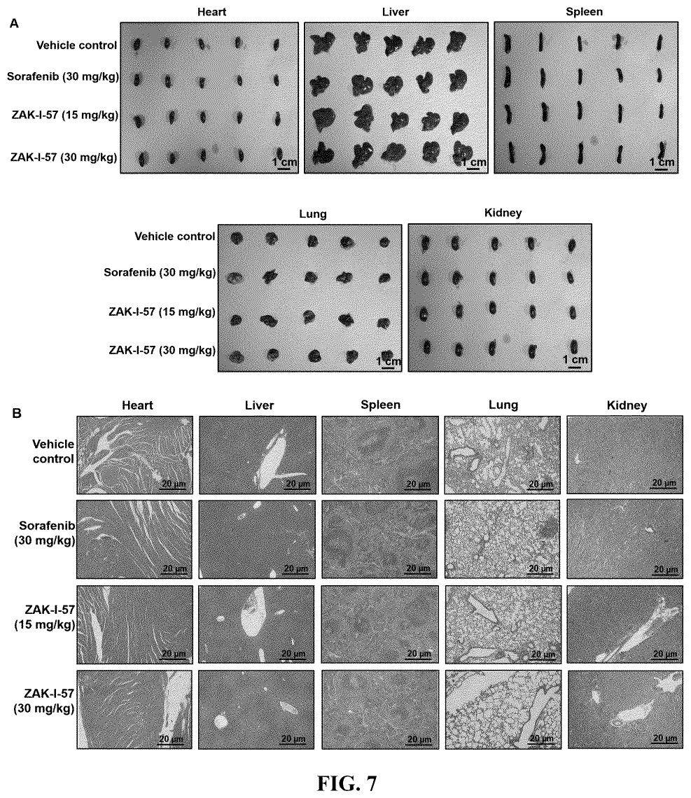

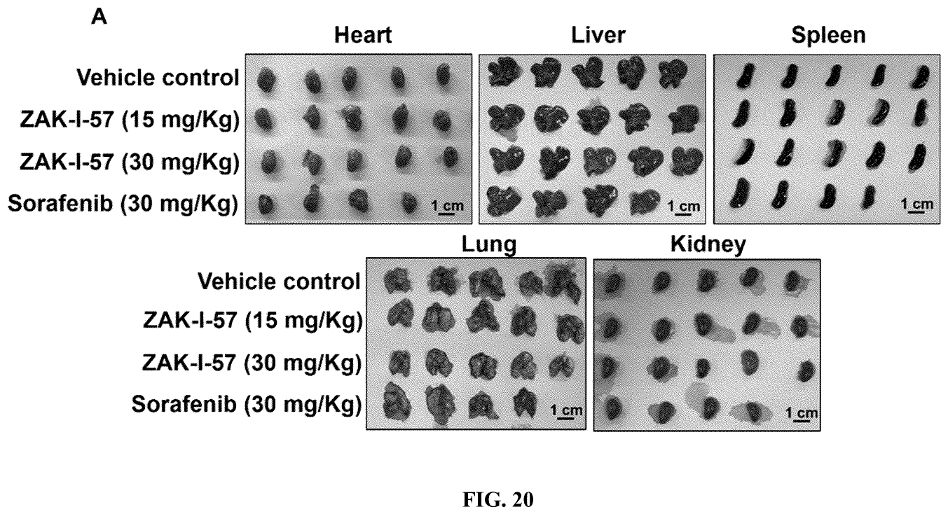

Comprehensive biosafety evaluations in both PLC/PRF/5 (FIG. 7) and PDTX (FIG. 20) mouse models confirmed the safety of ZAK-I-57 as a therapeutic agent for HCC. Gross anatomical examination of vital organs including hearts, livers, spleens, lungs, and kidneys across both models showed that ZAK-I-57 (15 and 30 mg/kg) and sorafenib (30 mg/kg) maintained tissue morphology comparable to the vehicle control, indicating minimal off-target toxicity (FIGS. 7A, 20A). Histopathological analysis further confirmed the absence of significant pathological changes in all treatment groups. In both PLC/PRF/5 model (FIG. 7B) and PDTX model (FIG. 20B), no histological abnormalities were observed in heart, liver, spleen, lung, or kidney tissues, reinforcing the safety of ZAK-I-57. These findings underscore ZAK-I-57's exceptional biosafety profile, positioning it as a promising therapeutic candidate for HCC.

The high molecular heterogeneity of HCC presents a formidable challenge in therapeutic intervention. Existing molecular-targeted agents, such as sorafenib and lenvatinib, have demonstrated limited efficacy due to single-target action, acquired resistance, and dose-limiting toxicities. To overcome these barriers, this study applies a polypharmacology-driven strategy to rationally design multi-target benzoxazinone derivatives, particularly ZAK-I-57, as a superior alternative to conventional tyrosine kinase inhibitors (TKIs). The integrative approach, combining systems pharmacology, molecular docking, in vitro and in vivo validation, and PDTX models, provides compelling evidence of ZAK-I-57's potential as a next-generation HCC therapy.

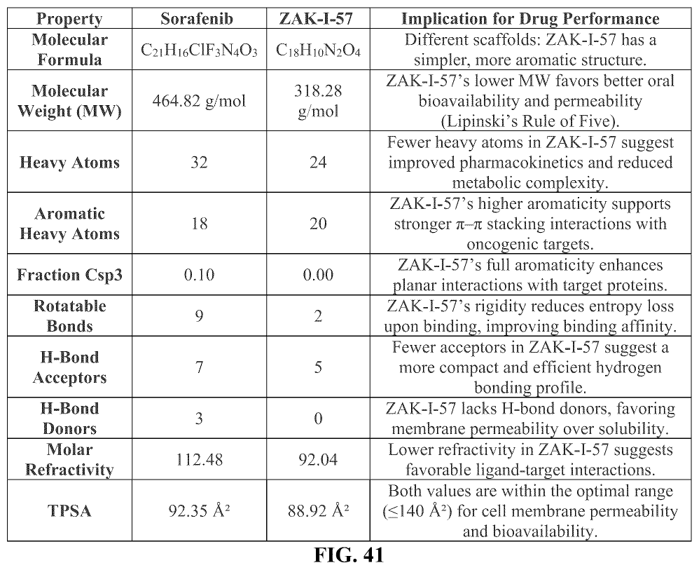

ZAK-I-57 exhibits structural and physicochemical attributes that offer significant pharmacological advantages over sorafenib, reinforcing its candidacy as an optimized therapeutic agent for HCC (FIG. 41). Its lower molecular weight (318.28 g/mol compared to 464.82 g/mol) enhances oral bioavailability and membrane permeability. The fully aromatic framework (Csp³=0.00) facilitates π-π stacking interactions, thereby strengthening target binding. Moreover, the compound's greater rigidity, characterized by only two rotatable bonds (versus nine in sorafenib), improves binding selectivity and stability. With a balanced polar surface area (TPSA: 88.92 Å²), ZAK-I-57 maintains optimal membrane permeability while ensuring effective target interactions. The absence of hydrogen bond donors (0 compared to 3 in sorafenib) reduces excessive polarity, favoring improved pharmacokinetics. Collectively, these structural refinements highlight ZAK-I-57's superior drug-like properties, bolstering its viability as a next-generation multi-targeted therapeutic for HCC.

Given the complex interplay of oncogenic signaling pathways in HCC, a multifaceted therapeutic approach is important. Using systems pharmacology, we identified 50 anti-HCC core targets, including EGFR, c-Myc, ERBB2, ESR1, CCND1, and HSP90AA1, all of which play critical roles in tumor initiation, proliferation, angiogenesis, and resistance mechanisms. The functional enrichment analysis revealed that these targets regulate kinase signaling, apoptotic pathways, and cellular stress responses, reinforcing the potential of benzoxazinone derivatives as broad-spectrum inhibitors. KEGG pathway analysis further highlighted that these targets contribute to EGFR tyrosine kinase inhibitor resistance, ErbB signaling, PI3K-Akt, and VEGF pathways, all of which are implicated in HCC aggressiveness and therapeutic resistance. Unlike single-target therapies, ZAK-I-57 inhibits multiple oncogenic nodes, reducing the probability of compensatory pathway activation, a common limitation of current TKIs.

To establish a structural basis for ZAK-I-57's multi-target efficacy, molecular docking was performed against six anti-HCC core targets. The high binding affinities of ZAK-I-57 toward EGFR (MET793, PR0794), c-Myc (LYS24, LYS45), and ERBB2 (ARG82, CYS234) suggest a robust inhibitory profile. The interaction at MET793 within EGFR's ATP-binding pocket suggests that ZAK-I-57 may function as a non-ATP-competitive inhibitor, a crucial distinction that reduces the likelihood of resistance mutations, a limitation seen with erlotinib-resistant HCC case. c-Myc, a key transcriptional regulator in HCC, lacks a traditional druggable pocket, yet ZAK-I-57 effectively interacts with LYS24 and LYS45, suggesting a potential disruption of Myc-Max dimerization, a critical step for oncogenic transcriptional activation. Given that c-Myc amplification is associated with poor prognosis in HCC and is a key driver of metabolic reprogramming and cell cycle progression, its inhibition represents a significant therapeutic advantage over conventional kinase inhibitors. Furthermore, ZAK-I-57's interaction with ERBB2 at ARG82 and CYS234 suggests disruption of dimerization-dependent activation. Unlike existing ERBB2 inhibitors, which focus on kinase domain inhibition, ZAK-I-57 appears to interfere with receptor dimerization and downstream oncogenic signaling, broadening its therapeutic potential.

Consistent with the molecular docking predictions, we further confirmed that ZAK-I-57 effectively suppressed expression of EGFR, c-Myc, and ERBB2 in cell-based model. In PLC/PRF/5 tumor-bearing mice and PDTX mouse models, ZAK-I-57 treatment (30 mg/kg) resulted in substantial downregulation of EGFR, c-Myc, and Bcl-2, with a concurrent increase in Bax expression (FIGS. 4F-4J and 5G-5K). Bcl-2 overexpression is a hallmark of HCC resistance to chemotherapy, as it inhibits mitochondrial outer membrane permeabilization (MOMP), preventing cytochrome c release and apoptotic cascade activation. The observed upregulation of Bax and concomitant downregulation of Bcl-2 provides strong evidence for the activation of the intrinsic apoptotic pathway, a crucial mechanism often suppressed in HCC. These findings suggest that ZAK-I-57 exerts both direct oncogenic inhibition and apoptotic reprogramming, reinforcing its potential as a comprehensive therapeutic strategy.

The in vitro cytotoxicity results demonstrated that ZAK-I-57 exhibits potent and selective cytotoxicity against HCC cells, with IC₅₀ values lower than sorafenib and artesunate. More importantly, ZAK-I-57 displayed minimal cytotoxicity in normal hepatocytes (MIHA cells, IC₅₀>100 μM), confirming its high selectivity. This selective cytotoxicity profile is crucial for reducing off-target toxicities, a major limitation of first-generation TKIs. Both PLC/PRF/5 tumor-bearing mice and PDTX mouse models demonstrated dose-dependent tumor suppression, with ZAK-I-57 at 30 mg/kg outperforming sorafenib in reducing tumor volume and weight. This superior efficacy is attributed to its simultaneous inhibition of multiple oncogenic drivers and apoptotic reprogramming. The reduction in Ki-67-positive cells further confirmed its strong antiproliferative effects, with the 30 mg/kg dose inducing significantly greater Ki-67 suppression than sorafenib. A major limitation of current HCC therapies is their systemic toxicity, leading to hepatic dysfunction, cardiovascular complications, and renal impairment. Histopathological analysis of vital organs (hearts, livers, spleens, lungs, kidneys) revealed no significant toxicological abnormalities in ZAK-I-57-treated mice, in stark contrast to sorafenib's reported hepatotoxicity. The absence of weight loss, organ damage, or significant biochemical alterations suggests that ZAK-I-57 possesses a superior therapeutic window compared to existing TKIs.

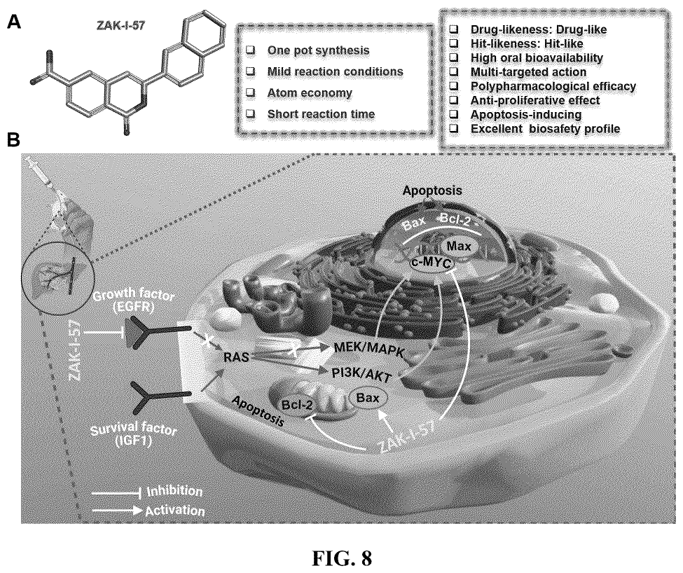

In summary, ZAK-I-57 is established as a highly potent multi-targeted therapeutic with superior efficacy, selectivity, and safety over existing HCC treatments. By concurrently disrupting key oncogenic pathways, modulating apoptotic regulators and demonstrating robust tumor suppression, it presents a significant advancement in molecular-targeted therapy (FIGS. 8A and 8B). Its polypharmacological design, strong translational potential, and excellent safety profile reinforce its viability for clinical development. While ZAK-I-57 demonstrates robust multi-target efficacy and a favorable safety profile, certain limitations remain. Potential adaptive resistance mechanisms require further investigation to evaluate long-term therapeutic effectiveness. Additionally, comprehensive pharmacokinetic and biodistribution studies are needed to elucidate its metabolic stability and clearance. Future studies will also focus on exploring combinatorial strategies with existing HCC therapies to enhance efficacy and mitigate potential resistance, supporting its clinical translation.

### EXAMPLES

### Materials

All reagents and solvents were obtained from commercial suppliers and used without further purification, unless specified otherwise.

### Synthesis of ZAK-I-55, ZAK-I-57, ZAK-I-64, ZAK-I-68, ZAK-I-87, ZAK-I-90, ZAK-I-93, and ZAK-I-97

A series of novel benzoxazinone derivatives, designated ZAK-I-55, ZAK-I-57, ZAK-I-64, ZAK-I-68, ZAK-I-87, ZAK-I-90, ZAK-I-93, and ZAK-I-97, were synthesized by reacting substituted 2-aminobenzoic acids (1) with substituted benzoyl chlorides (2 or 3) (FIG. 9). The reaction was performed under mild conditions (10-30° C.) in the presence of pyridine. Briefly, 0.91 g of 4-nitro anthranilic acid (1) (0.005 mol) in pyridine (20 ml) was added to the respective substituted benzoyl chlorides (2 and 3) (0.01 moles) under continuous stirring at 10° C. for ten minutes. The reaction mixture was then stirred at 30° C. for another half an hour. The reaction was monitored by thin-layer chromatography (TLC). After completion of the reaction, the product was poured onto ice-cold H₂O. The resultant precipitate was filtered, washed with ice-cold water, and dried in an oven at 50° C.

### 2-(4-bromophenyl)-7-nitro-4H-3,1-benzoxazin-4-one (ZAK-I-55)

Yield: 86%; Grey solid; mp: 118° C.; IR (νₘₐₓ, KBr, cm⁻¹): 1749, 1624; ¹H NMR (CDCl₃, 400 MHz) δ: 8.49 (d, 1H, Ar—H, J=2.1 Hz), 8.37 (d, 1H, Ar—H, J=8.2 Hz), 8.24 (dd, 1H, Ar—H, J=2.2, 8.6 Hz), 7.84 (t, 1H, Ar—H, J=7.2 Hz), 7.78-7.65 (m, 3H, Ar—H); ¹³C NMR (CDCl₃, 75 MHz) δ: 157.9, 155.8, 154.6, 152.5, 143.8, 134.6, 133.4, 132.6, 131.4, 129.6, 128.9, 122.4; ESI-MS: 346.02 [M-H]⁻; Anal. Calcd. For C₁₄H₇BrN₂O₄(MW: 347.10 g/mol): C, 48.44%; H, 2.03%; N, 8.07%; Found: C, 48.45%; H, 2.02%; N, 8.06%.

### 2-(naphthalen-2-yl)-7-nitro-4H-3,1-benzoxazin-4-one (ZAK-I-57)

Yield: 73%; Grey solid; mp: 92° C.; IR (νₘₐₓ, KBr, cm⁻¹):1751, 1625; ¹H NMR (CDCl₃, 400 MHz) δ: 8.56 (d,1H, Ar—H, J=2.3 Hz), 8.41 (d, 1H, Ar—H, J=8.0), 8.28 (dd, 1H, Ar—H, J=2.1, 8.2 Hz), 8.38-7.62 (m, 7H, Ar—H); ¹³C NMR (CDCl₃, 75 MHz) δ: 159.6, 156.4, 154.8, 152.4, 138.8, 134.6, 133.2, 132.4, 131.7, 131.5, 130.2, 129.6, 129.2, 129.0, 127.9, 125.6, 122.2, 117.4; ESI-MS: 317.12 [M-H]⁻; Anal. Calcd. For C₁₈H₁₀N₂O₄(MW: 318.25 g/mol): C, 67.92%; H, 3.17%; N, 8.80%; Found: C, 67.94%; H, 3.14%; N, 8.81%.

### 2-(2-methylphenyl)-7-nitro-4H-3,1-benzoxazin-4-one (ZAK-I-64)

Yield: 72%; Grey solid; mp: 79° C.; IR (νₘₐₓ, KBr, cm⁻¹):1751, 1619; ¹H NMR (CDCl₃, 400 MHz) δ: 8.49 (d, 1H, Ar—H, J=2.1 Hz), 8.37 (d,1H, Ar—H, J=8.2 Hz), 8.25 (dd, 1H, Ar—H, J=2.1, 8.4 Hz), 7.65 (d, 1H, Ar—H, J=8.1 Hz), 7.41-7.32 (m, 3H, Ar—H), 2.44 (s, 3H, CH₃); ¹³C NMR (CDCl₃, 75 MHz) δ: 159.9, 157.4, 154.6, 153.4, 141.2, 138.6, 131.5, 130.2, 129.2, 128.8, 128.6, 127.3, 124.7, 119.8, 20.1; ESI-MS: 281.08 [M-H]⁻; Anal. Calcd. For C₁₅H₁₀N₂O₄(MW: 282.15 g/mol): C, 63.83%; H, 3.57%; N, 9.92%; Found: C, 63.84%; H, 3.56%; N, 9.90%.

### 2-(4-hydroxyphenyl)-7-nitro-4H-3,1-benzoxazin-4-one (ZAK-I-68)

Yield: 83%; Yellow solid; mp: 90° C.; IR (νₘₐₓ, KBr, cm⁻¹): 3345, 1740, 1626; ¹H NMR (CDCl₃, 400 MHz) δ: 8.52 (d,1H, Ar—H, J=2.0 Hz), 8.39 (d,1H, Ar—H, J=8.4 Hz), 8.29 (dd,1H, Ar—H, J=2.0, 8.3 Hz), 7.90-7.81 (m, 2H, Ar—H), 7.07-6.99 (m, 2H, Ar—H), 4.22 (s, 1H, OH); ¹³C NMR (CDCl₃, 75 MHz) δ:158.8, 156.9, 154.4, 154.2, 152.3, 136.6, 132.4, 130.8, 128.5, 122.6, 120.3, 118.6; ESI-MS: 283.05 [M-H]⁻; Anal. Calcd. For C₁₄H₈N₂O₅ (MW: 284.21 g/mol): C, 59.16%; H, 2.84%; N, 9.86%; Found: C, 59.15%; H, 2.83%; N, 9.88%.

### 2-(3-fluorophenyl)-7-nitro-4H-3,1-benzoxazin-4-one (ZAK-I-87)

Yield: 79%; Yellow solid; mp: 104° C.; IR (νₘₐₓ, KBr, cm⁻¹): 1770, 1665; ¹H NMR (CDCl₃, 400 MHz) δ: 8.47 (d,1H, Ar—H, J=2.2 Hz), 8.39 (d, 1H, Ar—H, J=8.2), 8.24 (dd,1H, Ar—H, J=2.4, 8.7 Hz), 7.76-7.68 (m, 3H, Ar—H) 7.28 (m,1H, Ar—H); ¹³C NMR (CDCl₃, 75 MHz) δ: 164.3, 159.3, 154.4, 154.3, 152.6, 137.8, 132.2, 131.8, 130.2, 128.4, 124.2, 122.4, 119.6, 116.3; ESI-MS: 285.06 [M-H]-; Anal. Calcd. For C₁₄H₇FN₂O₄(MW: 286.19 g/mol): C, 58.75%; H, 2.47%; N, 9.79%; Found: C, 58.71%; H, 2.50%; N, 9.80%.

### 2-(3-chlorophenyl)-7-nitro-4H-3,1-benzoxazin-4-one (ZAK-I-90)

Yield: 75%; Brown solid; mp: 98° C.; IR (νₘₐₓ, KBr, cm⁻¹): 1758, 1628; ¹H NMR (CDCl₃, 400 MHz) δ: 8.53 (d, 1H, Ar—H, J=2.1 Hz), 8.29 (dd,1H, Ar—H, J=2.2, 8.6 Hz), 8.22-8.12 (m, 3H, Ar—H) 7.49-7.44 (m,2H, Ar—H); ¹³C NMR (CDCl₃, 75 MHz) δ: 159.6, 156.8, 154.6, 152.2, 141.5, 137.6, 133.6, 132.6, 131.3, 129.8, 129.5, 128.6; ESI-MS: 301.46 [M-H]-; Anal. Calcd. For C₁₄H₇ClN₂O₄(MW: 302.64 g/mol): C, 55.56%; H, 2.33%; N, 9.26%; Found: C, 55.53%; H, 2.35%; N, 9.27%.

### 2-[(E)-2-(4-fluorophenyl)ethenyl]-7-nitro-4H-3,1-benzoxazin-4-one (ZAK-I-93)

Yield: 68%; Grey solid; mp: 138° C.; IR (νₘₐₓ, KBr, cm⁻¹): 1780, 1660, 978; 1H NMR (CDCl₃, 400 MHz) δ: 8.55 (d,1H, Ar—H, J=2.2 Hz), 8.37 (d, 1H, Ar—H, J=8.1), 8.20 (dd,1H, Ar—H, J=2.2, 8.2 Hz), 7.79 (d, 1H, CH═CH, J=16.4 Hz), 7.63-7.32 (m, 4H, Ar—H), 6.76 (d, 1H, CH═CH, J=16.2 Hz); ¹³C NMR (CDCl₃, 75 MHz) δ:164.7, 159.5, 157.2, 156.4, 154.6, 147.1, 141.8, 136.4, 130.2, 128.1, 126.8, 119.2, 116.9, 116.4; ESI-MS: 311.04 [M-H]-; Anal. Calcd. For C₁₆H₉FN₂O₄(MW: 312.21 g/mol): C, 61.54%; H, 2.91%; N, 8.97%; Found: C, 61.55%; H, 2.89%; N, 8.98%.

### 2-[(E)-2-(3,4-dimethoxyphenyl)ethenyl]-7-nitro-4H-3,1-benzoxazin-4-one (ZAK-I-97)

Yield: 63%; Brown solid; mp: 152° C.; IR (νₘₐₓ, KBr, cm¹):1764, 1651, 969; ¹H NMR (CDCl₃, 400 MHz) δ: 8.57 (d, 1H, Ar—H, J=2.2 Hz), 8.36 (d, 1H, Ar—H, J=8.0 Hz), 8.19 (dd, 1H, Ar—H, J=2.2, 8.4 Hz), 7.74 (d, 1H, CH═CH, J=16.3 Hz), 7.23-7.09 (m, 3H, Ar—H), 6.79 (d, 1H, CH═CH, J=16.0 Hz), 3.90 (s, 6H, OCH₃); ¹³C NMR (CDCl₃, 75 MHz) δ: 158.6, 157.8, 156.4, 156.2, 152.4, 146.9, 136.0, 133.1, 132.5, 131.2, 128.3, 127.1, 120.4, 120.3, 116.6, 112.0, 56.1; ESI-MS: 353.18 [M-H]-; Anal. Calcd. For C₁₈H₁₄N₂O₆ (MW: 354.31 g/mol): C, 61.02%; H, 3.98%; N, 7.91%; Found: C, 61.01%; H, 3.98%; N, 7.92%.

### Cell Culture

Huh7 and PLC/PRF/5 (CRL-8024™) cells were purchased from Thermo Fisher Scientific and the ATCC, respectively. MIHA was kindly provided by Dr. J. R. Chowdhury, Albert Einstein College of Medicine, New York. The cell lines were cultured in Dulbecco's modified Eagle medium (DMEM, Gibco, MA, USA) supplemented with 10% fetal bovine serum (FBS, BI, MA, USA) and 1× penicillin-streptomycin solution (Solarbio) at 37° C. in a humid, 5% CO₂ atmosphere.

### MTT Assay

Huh7 and PLC/PRF/5 cells at a density of 5×10³ cells/well were cultured in 96-well plates and incubated at 37° C. in 5% CO₂. Both cell lines were treated with different concentrations of benzoxazinone derivatives (ZAK-I-57, ZAK-I-64, and ZAK-I-68). Both cell lines were incubated with ZAK-I-57 for 24 h and for 48 h with ZAK-I-64 and ZAK-I-68. MTT (3-[4,5-dimethylthiazol-2-yl]-2,5-diphenyltetrazolium bromide) solution was then added to each well and incubated for 3 h in a CO₂ atmosphere at 37° C. Insoluble formazan crystals were dissolved by adding 100 μL of dimethyl sulfoxide (DMSO) to each well, followed by orbital agitation for 10 min. A minimum of three biological replicates were analyzed using a plate reader to determine IC₅₀. The same experiment was performed with MIHA hepatocyte cells to evaluate the selectivity cytotoxicity of ZAK-I-57.

### Animals

Male BALB/c nude mice (6-8 weeks old) were obtained from the Centralized Animal Facilities at the Hong Kong Polytechnic University. All animals were bred and housed under specific pathogen-free conditions, with access to sterile food and water. Environmental conditions were strictly controlled, with temperature maintained at 23±2° C., relative humidity between 30%-70%, and a 12-hour light/dark cycle. Mice were group-housed in accordance with institutional guidelines for recommended stocking density. The animal experiments were conducted according to institutional guidelines, and the experimental procedures were approved by the PolyU Animal Experimentation Ethics Committee (Ref. No. 19-20/57-ABCT-R-STUDENT).

### PLC/PRF/5 Tumor-Bearing Mouse Model

PLC/PRF/5 cells (5×10⁵) were suspended in 100 μL PBS with Matrigel (1:1 ratio) and injected into the right back of nude mice. Small tumors (<70 mm³) were formed two weeks after cell inoculation. Mice were then randomized into the vehicle control group, ZAK-I-57 treated groups (15 mg/kg and 30 mg/kg), or sorafenib (30 mg/kg) groups. Mice were administered 200 μL of vehicle water and drugs by oral gavage daily for three weeks. Tumor size was measured twice a week, and tumor volumes were calculated using the formula [(length×width×depth)/2 mm³] while each mouse was weighed. At the end of the experiment, the mice were sacrificed, and the tumors and organs were collected for further experiments.

### Patient-Derived Tumor Xenograft (PDTX) Mouse Model

The procedure for the establishment of PDTX #1 was previously described. Tumor cells from PDTX #1 was inoculated subcutaneously on the back of nude mice. When the tumors reached approximately 1,000 mm³, mice with the first generation of xenografts (P1) were sacrificed, and the xenografts were isolated and expanded for the second generation (P2). When P2 xenografts reached an average volume of 70 mm³, mice were subjected to ZAK-I-57 and sorafenib treatment. The development of PDTX was approved by the Institutional Review Board of the University of Hong Kong/Hospital Authority Hong Kong West Cluster (UW 17-056) and informed consents were obtained from patients.

### Western Blot Assay

The Huh7 cells and tumor tissues were lysed on ice using the lysis buffer. Protein concentration was quantified using Bradford assay (Bio-Rad, Hercules, CA, USA). Equivalent amounts of protein (25-50 μg) were loaded onto 10% sodium dodecyl sulfate-polyacrylamide gel electrophoresis (SDS-PAGE) and transferred to polyvinylidene fluoride (PVDF) membranes. Membranes were blocked with 5% non-fat milk in Tris-buffered saline Tween 20 (TBST) for an hour. The blots were incubated with primary antibodies (EGFR (4267S, Cell signaling technology, MA, USA), c-Myc (5605S, Cell signaling technology, MA, USA), Bax (2772S, Cell signaling technology, MA, USA), Bcl-2 (sc-7382, Santa Cruze, TX, USA), ERBB2 (2165S, Cell signaling technology, MA, USA), and α-tubulin (62204, Invitrogen, MA, USA), 1:1000) at 4° C. overnight. Subsequently, the membranes were washed thrice (15 min each) with TBST solution and incubated for 1 h with secondary horseradish peroxidase-conjugated antibodies (NA934 and NA931, Cytiva, MA, USA, 1:5000). Blots were then detected using an ECL kit and photographed using a ChemiDoc Imaging System (Bio-Rad, MA, USA). The bands were quantified using the ImageJ software (NIH, USA). The intensities of the bands for each protein sample were normalized to those of α-tubulin (internal standard protein). Quantitative data are presented as a fold change of untreated control.

### Immunohistochemistry (IHC) Staining Assay

Tumors were fixed in 4% formalin, embedded in paraffin, and sectioned at 5 m thickness for IHC staining. Ki-67 primary antibody (ab16667, Abcam, MA, USA) and anti-mouse IgG recombinant secondary antibody (7056S, Cell Signaling Technology, MA, USA) were used for IHC staining. Hemotoxin (Invitrogen, MA, USA) was used to stain nuclei. The number of positively stained cells in Ki-67-stained slides was evaluated using ImageJ software (NIH, MD, USA).

### Hematoxylin & Eosin (H&E) Staining Assay

H&E staining was performed on histological sections of organs (heart, spleen, kidney, liver, and lung) to examine the toxicity of ZAK-I-57 in PLC/PRF/5 tumor-bearing and PDTX model mice. Briefly, the histological slides were deparaffinized using 100% xylene (v/v). The slides were then washed several times with ethanol solutions of various concentrations (70-100%, v/v) and immersed in hematoxylin for 5 min, followed by a quick dip in acidic alcohol. Next, the slides were immersed in Scott's tap water for 3 min and in 1% eosin for 30 s. Finally, the slides were dehydrated using ethanol solutions of various concentrations (70%-100%, v/v) and 100% xylene (v/v) and mounted using a mounting medium.

### Statistical Analysis

All in vitro data are presented as mean±SD, whereas in vivo data are expressed as the mean±S.E.M.. Quantitative results were analyzed using one-way analysis of variance (ANOVA). Statistical significance was set at p<0.05. All statistical analyses were performed using Prism 5 software (GraphPad, CA, USA).

### DFT Calculations

The geometry of the chemotherapeutic drugs (ZAK-I-55, ZAK-I-57, ZAK-I-64, ZAK-I-87, ZAK-I-90, ZAK-I-93,) were optimized in this study utilizing DFT simulations conducted throughout the Gaussian 16 software with basis set B3LYP/6-31G. We selected this basis set because it is significantly related to the geometry optimization of such molecules. Relaxation of the structural features is the first step in our computation.

### Physicochemical, Pharmacokinetics, Drug-Likeness, and Medicinal Chemistry Properties Analysis

The physicochemical, pharmacokinetic, drug-likeness, and medicinal chemistry properties of benzoxazinone derivatives were proposed using the SwissADME online web tool.

### Prediction of Potential Targets of Benzoxazinone Derivatives

Potential protein targets of benzoxazinone derivatives (ZAK-I-55, ZAK-I-57, ZAK-I-64, ZAK-I-68, ZAK-I-87, ZAK-I-90, ZAK-I-93, and ZAK-I-97) were predicted using the SwissTargetPrediction online tool. After merging the datasets, 638 potential protein targets were identified with a probability score >0. Since different compounds can share common targets, redundancy was removed by eliminating duplicates, ensuring each protein target was counted only once. This refinement resulted in 265 unique potential protein targets for further analysis.

### HCC-Related Target Screening

HCC-related targets were retrieved from two databases, OncoDB.HCC (oncodb.hcc.ibms.sinica.edu.tw) and Liverome (liverome.kobic.re.kr/index.php).

### Identification of Intersection Targets

Intersection targets were identified between the potential targets of benzoxazinone derivatives and HCC-related targets using the VENNY 2.1 online tool.

### Protein-Protein Interaction (PPI) Analysis

PPI analysis was further carried out on identified intersected targets at a medium confidence score of 0.400 and species limited to “Homo sapiens” by employing the STRING database. The results of the STRING PPI analysis were further uploaded to the Cytoscape software (version 3.9.0, Boston, MA, USA) in a. tsv file format for further analysis to determine the potential anti-HCC core targets.

### Expression of Anti-HCC Core Targets in Liver Hepatocellular Carcinoma (LHIC) Analysis

The expression of the top six anti-HCC core targets in LHIC was analyzed using the GEPIA database.

### Network Construction Between Anti-HCC Targets and Benzoxazinone Derivatives

The network between anti-HCC targets and benzoxazinone derivatives was further constructed by employing Cytoscape software (version 3.9.0, Boston, MA, USA).

### GO and KEGG Enrichment Analysis

The GO and KEGG enrichment analysis were further performed on fifty intersected targets by employing the database for annotation, visualization, and integrated discovery (DAVID; Version 6.8) (david.ncifcrf.gov/). The GO terms were categorized into three types: cellular component (CC), biological process (BP), and molecular function (MF). By uploading the data to the Bioinformatics platform (www.bioinformatics.com.cn/), the top 10 GO analysis data (BP, CC, and MF) and top 30 KEGG pathways were further shown in the form of an enrichment dot bubble plot. The classical hypergeometric test was used to determine statistical significance. The adjusted p≤0.05 was utilized as the significant threshold in our investigation.

### Molecular Docking

Two-dimensional (2D) structures of benzoxazinone derivatives were generated using ChemDraw Ultra (version 12.0) and saved in a MOL file (.mol) format. Three-dimensional (3D) structures were generated and saved in PDB format by uploading each MOL file (.mol) to BIOVIA Discovery Studio Visualizer software. The Protein Data Bank (www.rcsb.org/) was used to obtain the crystal structures of the anti-HCC core targets (c-Myc 5I4Z, ESR1 1R5K, EGFR 5Y9T, HSP90AA1 4BQG, CCND1 5VZU, and ERBB2 2A91). The ligands and water molecules from each targeted protein were hauled out, and subsequently, grid construction was also performed using the BIOVIA Discovery Studio Visualizer software. PDB files were uploaded to AutoDock Vina (version 1.2.0). and receptor proteins were charged with Kollman and Gasteiger partial charges. Benzoxazinone derivatives were uploaded to AutoDock Vina (version 1.2.0.) in PDB format. AutoDock Vina (version 1.2.0.) was used to convert both proteins and benzoxazinone derivatives to the pdbqt format. Subsequently, proteins and benzoxazinone derivatives in pdbqt format were used to write scripts for molecular docking using AutoDock Vina (version 1.2.0.), and docked complex findings were acquired. The docked complexes were further analyzed to determine the binding capabilities of the molecules' and targets' through various interactions using the BIOVIA Discovery Studio Visualizer software. A binding energy <0 implies that a ligand may instinctively bind to the receptor. It is commonly recognized that the lower the energy score of the ligand and receptor binding configuration, the more probable the binding will occur.

What is claimed is:

1. A method of treating hepatocellular carcinoma in a subject in need thereof, the method comprising: administering a therapeutically effective amount of a compound to the subject, wherein the compound has Formula 1: 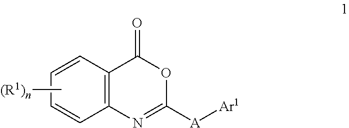
or a pharmaceutically acceptable salt thereof, wherein:
m is a whole number selected from 0-4;
A is —CH₂CH₂, —CH═CH—, —C≡C—, or absent;
Ar¹ is aryl or heteroaryl;
R¹ for each instance is independently alkyl, haloalkyl, perhaloalkyl, alkenyl, alkynyl, cycloalkyl, heterocycloalkyl, aryl, aralkyl, heteroaryl, nitrile, nitro, azido, —OR, —SR, —NR₂, —(C═O)R, —(C═O)OR, —(C═O)NR₂, —(C═NR)NR₂, —(S═O)R, —S(O)₂R, —S(O)₂OR, —S(O)₂NR₂, —(P═O)(OR)₂, or —(CR²₂)ₚY, wherein p for each occurrence is a whole number selected from 1-10; R² for each occurrence is independently selected from the group consisting of hydrogen, alkyl, alkenyl, alkynyl, cycloalkyl, heterocycloalkyl, aryl, and heteroaryl; and Y for each occurrence is selected from the group consisting of OR, SR, NR₂, —(C═O)R, —(C═O)OR, —O(C═O)R, —O(C═O)OR, —(C═O)NR₂, —(NR)(C═O)R, —(NR)(C═O)OR, —O(C═O)NR₂, —O(C═NR)NR₂, —(NR)(C═O)NR₂, —(C═NR)NR₂, —(NR)(C═NR)NR₂, —(S═O)R, —S(O)₂R, —S(O)₂OR, —S(O)₂NR₂, —OS(O)₂R, —(NR)S(O)₂R, —OS(O)₂OR, —OS(O)₂NR₂, —(NR)S(O)₂NR₂, —(NR)S(O)₂OR, and —(P═O)(OR)₂; and
R for each instance is independently selected from the group consisting of hydrogen, alkyl, haloalkyl, perhaloalkyl, alkenyl, alkynyl, cycloalkyl, heterocycloalkyl, aryl, heteroaryl, and aralkyl; or two instances of R together with the atom they are covalently bonded form a 3-6 membered cycloalkyl or heterocyloalkyl.

2. The method of claim 1, wherein A is —CH═CH— or absent.

3. The method of claim 1, wherein Ar¹ is a moiety selected from the group consisting of: 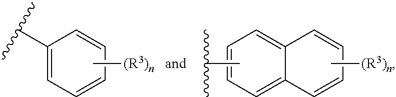
wherein n is a whole number selected from 0-4;
R³ for each instance is independently alkyl, haloalkyl, perhaloalkyl, alkenyl, alkynyl, cycloalkyl, heterocycloalkyl, aryl, aralkyl, heteroaryl, nitrile, nitro, azido, —OR, —SR, —NR₂, —(C═O)R, —(C═O)OR, —(C═O)NR₂, —(C═NR)NR₂, —(S═O)R, —S(O)₂R, —S(O)₂OR, —S(O)₂NR₂, —(P═O)(OR)₂, or —(CR²₂)ₚY, wherein p for each occurrence is a whole number selected from 1-10; R² for each occurrence is independently selected from the group consisting of hydrogen, alkyl, alkenyl, alkynyl, cycloalkyl, heterocycloalkyl, aryl, and heteroaryl; and Y for each occurrence is selected from the group consisting of OR, SR, NR₂, —(C═O)R, —(C═O)OR, —O(C═O)R, —O(C═O)OR, —(C═O)NR₂, —(NR)(C═O)R, —(NR)(C═O)OR, —O(C═O)NR₂, —O(C═NR)NR₂, —(NR)(C═O)NR₂, —(C═NR)NR₂, —(NR)(C═NR)NR₂, —(S═O)R, —S(O)₂R, —S(O)₂OR, —S(O)₂NR₂, —OS(O)₂R, —(NR)S(O)₂R, —OS(O)₂OR, —OS(O)₂NR₂, —(NR)S(O)₂NR₂, —(NR)S(O)₂OR, and —(P═O)(OR)₂.

4. The method of claim 2 wherein n is 0 or 1 and R³ for each instance is independently alkyl, haloalkyl, perhaloalkyl, cycloalkyl, heterocycloalkyl, aryl, aralkyl, heteroaryl, nitrile, nitro, —OR, —SR, —NR₂, —(C═O)NR₂, —(NR)(C═O)R, or —(NR)(C═O)OR.

5. The method of claim 2 wherein n is 0 or 1 and R³ for each instance is independently alkyl, halide, or —OR.

6. The method of claim 1, wherein Ar¹ is selected from the group consisting of 4-bromophenyl, 4-hydroxy-phenyl, 2-methyl-phenyl, 3-chloro-phenyl, 4-fluoro-phenyl, 3,4-dimethoxy-phenyl, or 2-naphthyl.

7. The method of claim 1, wherein R¹ for each instance is independently alkyl, haloalkyl, perhaloalkyl, cycloalkyl, heterocycloalkyl, aryl, aralkyl, heteroaryl, nitrile, nitro, —OR, —SR, —NR₂, —(C═O)NR₂, —(NR)(C═O)R, or —(NR)(C═O)OR.

8. The method of claim 1, wherein at least one R¹ is nitro.

9. The method of claim 1, wherein the compound has Formula 2: 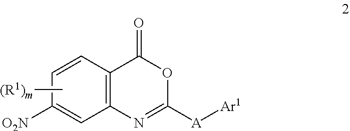
or a pharmaceutically acceptable salt thereof, wherein
m is a whole number selected from 0-2;
A is —CH═CH— or absent;
Ar¹ is a moiety selected from the group consisting of: 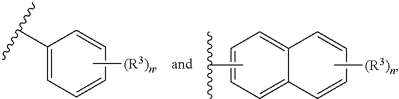
wherein n is a whole number selected from 0-2;
R¹ for each instance is independently alkyl, haloalkyl, perhaloalkyl, cycloalkyl, heterocycloalkyl, aryl, aralkyl, heteroaryl, nitrile, nitro, —OR, —SR, —NR₂, —(C═O)NR₂, —(NR)(C═O)R, or —(NR)(C═O)OR;
R³ for each instance is independently alkyl, haloalkyl, perhaloalkyl, cycloalkyl, heterocycloalkyl, aryl, aralkyl, heteroaryl, nitrile, nitro, —OR, —SR, —NR₂, —(C═O)NR₂, —(NR)(C═O)R, or —(NR)(C═O)OR; and
R for each instance is independently selected from the group consisting of hydrogen, alkyl, haloalkyl, perhaloalkyl, alkenyl, alkynyl, cycloalkyl, heterocycloalkyl, aryl, heteroaryl, and aralkyl; or two instances of R together with the atom they are covalently bonded form a 3-6 membered cycloalkyl or heterocyloalkyl.

10. The method of claim 9, wherein at least one R³ is selected from the group consisting of halide, —OR, and alkyl.

11. The method of claim 9, wherein Ar¹ is selected from the group consisting of 4-bromophenyl, 4-hydroxy-phenyl, 2-methyl-phenyl, 3-chloro-phenyl, 4-fluoro-phenyl, 3,4-dimethoxy-phenyl, and 2-naphthyl.

12. The method of claim 9, wherein m is 0 and at least one R³ is selected from the group consisting of halide, —OR, and alkyl.

13. The method of claim 1, wherein the compound is selected from the group consisting of: 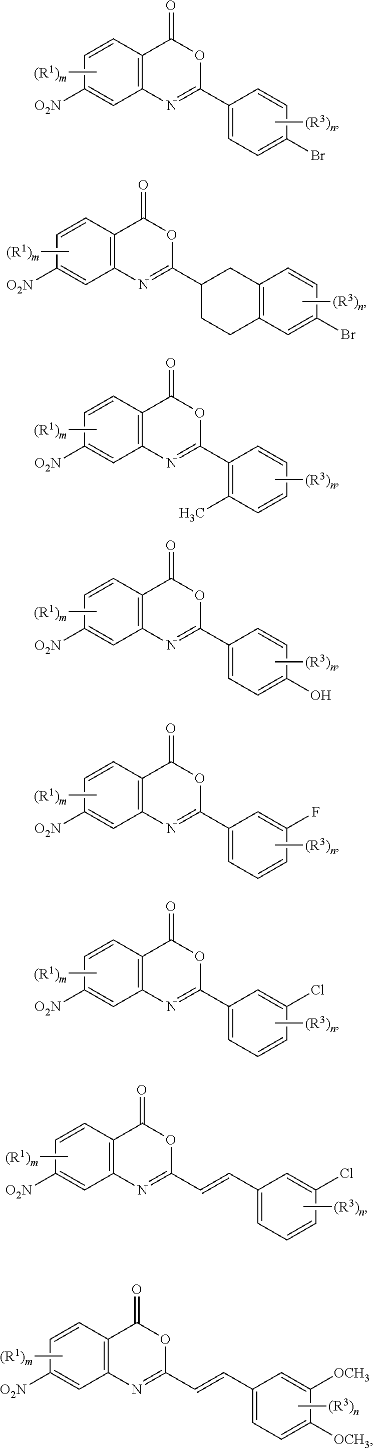
and
pharmaceutically acceptable salts thereof, wherein
m is a 0 or 1;
n is 0 or 1;
R¹ is alkyl, haloalkyl, perhaloalkyl, cycloalkyl, heterocycloalkyl, aryl, aralkyl, heteroaryl, nitrile, nitro, —OR, —SR, —NR₂, —(C═O)NR₂, —(NR)(C═O)R, or —(NR)(C═O)OR; and
R³ is alkyl, haloalkyl, perhaloalkyl, cycloalkyl, heterocycloalkyl, aryl, aralkyl, heteroaryl, nitrile, nitro, —OR, —SR, —NR₂, —(C═O)NR₂, —(NR)(C═O)R, or —(NR)(C═O)OR; and
R for each instance is independently selected from the group consisting of hydrogen, alkyl, cycloalkyl, heterocycloalkyl, aryl, heteroaryl, and aralkyl; or two instances of R together with the atom they are covalently bonded form a 3-6 membered cycloalkyl or heterocyloalkyl.

14. The method of claim 1, wherein the compound is selected from the group consisting of: 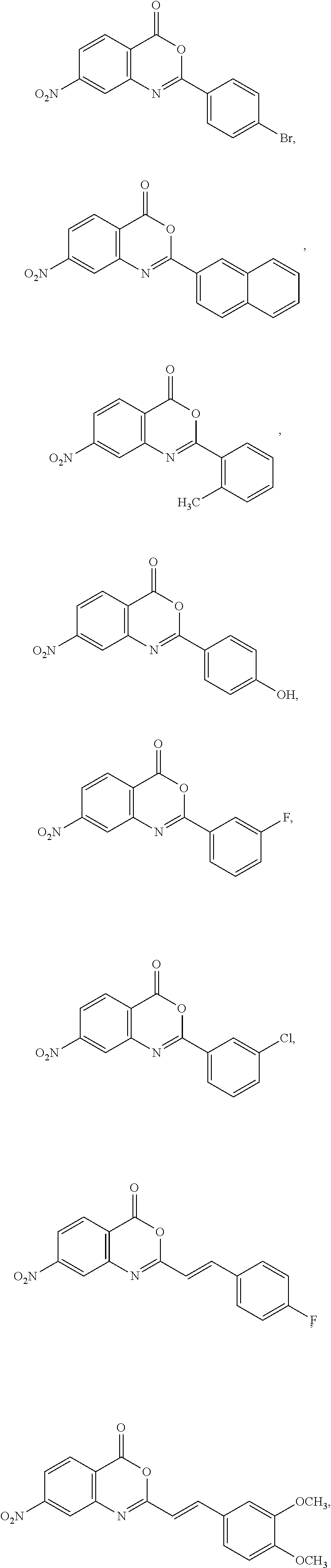
and
pharmaceutically acceptable salts thereof.

15. The method of claim 1, wherein the compound is:

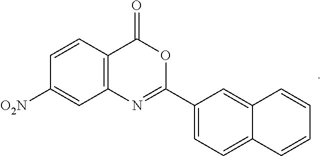 [SMILES: O=c1oc(-c2ccc3ccccc3c2)nc2cc([N+](=O)[O-])ccc12 ; "."]

16. The method of claim 1, wherein administration of the compound reduces the expression of one or more oncogenic proteins in the hepatocellular carcinoma, wherein the one or more oncogenic proteins is selected from the group consisting of EGFR, c-Myc, and ERBB2.

17. The method of claim 1, wherein administration of the compound results in at least one of an increase in expression of Bax and reduction in expression of Bcl-2 in the hepatocellular carcinoma.

18. The method of claim 1, wherein the compound induces apoptosis in the hepatocellular carcinoma.

19. The method of claim 1, wherein the compound exhibits selective cytotoxicity against the hepatocellular carcinoma.

20. The method of claim 1 wherein the compound reduces tumor volume and weight in a PLC/PRF/5 xenograft model.

21. The method of claim 1, wherein the compound reduces hepatocellular carcinoma progression in a patient-derived tumor xenograft (PDTX) model.

22. The method of claim 1, wherein the compound demonstrates anti-hepatocellular carcinoma efficacy comparable to or greater than sorafenib.

23. The method of claim 1, wherein the administration of the compound results in suppression of hepatocellular carcinoma proliferation as measured by Ki-67 expression.

24. The method of claim 1, wherein the compound does not induce observable histopathological alterations in the liver, kidney, heart, lung, or spleen of the subject, as determined by hematoxylin and eosin (H&E) staining.
